(19) United States
(12) Patent Application Publication
(10) Pub. No.: US 2024/0245339 A1
(43) Pub. Date: Jul. 25, 2024

(54) A Controller, Electronics Module, System and Method

(71) Applicant: Prevayl Innovations Limited, Manchester, Greater Manchester (UK)

(72) Inventor: Michael John Lynch, Cheshire (GB)

(21) Appl. No.: 17/999,225

(22) PCT Filed: Jun. 23, 2022
(86) PCT No.: PCT/GB2022/051599

§ 371 Date: Nov. 18, 2022

(30) Foreign Application Priority Data
Jun. 25, 2021 (GB) 2109156.6
Oct. 4, 2021 (GB) 2114184.1

(51) Int. Cl. A61B 5/332; A61B 5/00; A61B 5/333

(52) U.S. Cl. CPC A61B 5/332; A61B 5/333; A61B 5/6804; A61B 5/6843; A61B 2560/0209

(57) ABSTRACT

The controller is operable to enter a first measurement mode (S101). In the first measurement mode, the controller obtains contextual data indicative of whether a wearer of the wearable article is in a first motion state or a second motion state representative of a higher degree of activity of the wearer than the first motion state (S102). The controller determines from the contextual data whether the wearer of the wearable article is in the first motion state (S103). In response to the wearer being in the first motion state, the controller performs a measurement using a sensor of the wearable article (S104). The controller therefore selectively performs measurements based on the motion state of the wearer and may only perform measurements in the first measurement mode when the wearer is in the first motion state.

The present invention is directed towards a controller, electronics module, system and method. More particularly, the controller is suitable for use with a wearable article comprising sensors and controls the operation of the sensors of the wearable article based on the motion state of the wearer.

### BACKGROUND

Wearable articles, such as garments, incorporating sensors are wearable electronics used to measure and collect information from a wearer. Such wearable articles are commonly referred to as ‘smart clothing’. It is advantageous to measure biosignals of the wearer during exercise, or other scenarios.

It is known to provide a garment, or other wearable article, to which an electronic device (i.e. an electronic module, and/or related components) is attached in a prominent position, such as on the chest. Advantageously, the electronic device is a detachable device. The electronic device is configured to process the incoming signals, and the output from the processing is stored and/or displayed to a user in a suitable way.

A sensor senses biosignals such as electrocardiogram (ECG) signals and the biosignals are coupled to the electronic device, via an interface.

The sensors may be coupled to the interface by means of conductors which are connected to terminals provided on the interface to enable coupling of the signals from the sensor to the interface.

Electronics modules for wearable articles such as garments are known to communicate with user electronic devices over wireless communication protocols such as Bluetooth® and Bluetooth® Low Energy. These electronics modules are typically removably attached to the wearable article, interface with internal electronics of the wearable article, and comprise a Bluetooth® antenna for communicating with the user electronic device.

The electronics module includes drive and sensing electronics comprising components and associated circuitry, to provide the required functionality.

The drive and sensing electronics include a power source to power the electronic device and the associated components of the drive and sensing circuitry.

ECG sensing is used to provide a plethora of information about a person's heart. It is one of the simplest and oldest techniques used to perform cardiac investigations. In its most basic form, it provides an insight into the electrical activity generated within heart muscles that changes over time. By detecting and amplifying these differential biopotential signals, a lot of information can be gathered quickly, including the heart rate.

Typically, the detected ECG signals can be displayed as a trace to a user for information. Alternatively, or in addition to a signal trace, information can be derived from raw ECG signals through digital signal processing and displayed or presented to the user in other ways, for example such as simple hear rate figures in beats per minute.

The trace and/or the additional information can be displayed or presented to a user on a user electronic device such as a mobile phone. Within the context of this invention the user can be a wearer of the electronics module of any other user of the electronics module.

The optimum position for ECG sensing is to have the sensors positioned on either side of the heart of the wearer such that a differential voltage measurement can be performed as close the heart as possible where the voltage signal is strongest. Additional sensors can also be included at different positions although ECG sensing using only two sensors is preferred as it reduces the complexity of the wearable article.

In addition to the positioning of the sensors optimum sensing is performed when the sensors are held tightly against the skin surface of the wearer. This can be achieved using an adhesive conductive gel layer used in so-called “wet” electrodes although this is generally undesirable outside of clinical setting as the adhesive layer is uncomfortable to wear and not reusable.

Tight contact can also be achieved by using the wearable article to apply compression against the sensors to hold them in contact with the skin. Such wearable articles are typically in a form of chest-straps. This form of dry connection does not require an adhesive conductive medium, however, the tight-fitting nature of the wearable article means that they are typically only worn in fitness settings rather than as everyday articles as they are not comfortable to wear over long periods of time and may not have a desirable aesthetic for the wearer.

It is an object of the present disclosure to provide mechanisms for performing effective sensing, such as for measuring an ECG, using a wearable article without requiring the sensors to be positioned close to the heart of the wearer and/or continuously held tightly in contact with the skin surface.

It is another object of the present disclosure to provide improved constructions of wearable articles incorporating sensors particularly for wearable articles that are intended to be worn as daywear/night wearer rather than athletic performance clothing.

### SUMMARY

According to the present disclosure there is provided a controller, electronics module, system and method as set forth in the appended claims. Other features of the invention will be apparent from the dependent claims, and the description which follows.

According to a first aspect of the present disclosure, there is provided a controller for a wearable article. The controller is operable to enter a first measurement mode. In the first measurement mode, the controller is operable to: obtain contextual data indicative of whether a wearer of the wearable article is in a first motion state or a second motion state representative of a higher degree of activity of the wearer than the first motion state; determine from the contextual data whether the wearer of the wearable article is in the first motion state; and in response to the wearer being in the first motion state, perform a measurement using a sensor of the wearable article.

Advantageously, the controller uses contextual data to determine the motion state of the wearer and triggers the performance of a measurement using the sensor according to the determined motion state. If the motion state is a first, low, motion state, the controller performs a measurement. Otherwise, the controller may not perform a measurement. In this way, measurements may only be performed when wearer motion is low meaning that significant motion artefacts are unlikely to be present in the measured signal. Effective sensing can therefore be performed without the sensors being required to be located close to the chest region of the wearer or in tight contact with the body surface of the wearer. Moreover, selectively performing measurements based on the motion state of the wearer reduces power and memory consumption as the controller is not activated to continuously perform measurements when many of the measurements may be unusable due to having an undesirable amount of motion artefacts.

The sensor may be arranged to perform a measurement from a body surface of the wearer of the wearable article. The sensor may be an electrode such as an electrode used for potential (e.g. ECG) and/or impedance measurements.

In the first measurement mode, the controller may only be operable to perform a measurement using the sensor of the wearable article when the wearer is in the first motion state.

In response to the wearer being in the first motion state, the controller may be operable to store the measurement in a memory of the wearable article. In the first measurement mode, the controller may only be operable to store the measurement when the wearer is in the first motion state.

In response to the wearer being in the first motion state, the controller may be operable to perform a plurality of measurements using a sensor of the wearable article over a first predetermined time period. If the wearer remains in the first motion state during the first predetermined time period, the controller may be further operable to store the measurements. The measurements may be stored in an internal memory of the controller or a separate memory of the wearable article.

Advantageously, the controller may only store the plurality of measurements performed over the first predetermined time period if the wearer remains in the low motion state during the first predetermined time period. If the wearer transitions to a higher motion state during the first predetermined time period then the measurements may instead be disregarded. This avoids unnecessary memory consumption associated with storing inaccurate or unreliable measurement data. The controller is therefore able to perform monitoring of the wearer over an extended time period without requiring data to be deleted from the memory or offloaded to an external device. In this way, the controller is not required to activate a communicator of the wearable article to transmit measurement data to an external device. This can reduce power consumption.

The measurements may be performed at a fixed or variable measurement/sampling rate. Example measurement rates include 100 Hz, 200 Hz, 300 Hz, 400 Hz, 500 Hz.

The controller may be operable to determine from contextual data whether the wearer remains in the first motion state during the first predetermined time period.

If the wearer transitions from the first motion state to the second motion state during the first predetermined time period, the controller may be operable to discard the measurements performed during the first predetermined time period.

The controller may be operable to determine from the contextual data whether the wearer of the wearable article has been in the first motion state for more than a first predetermined time period. In response to the wearer being in the first motion state for more than the first predetermined time period, the controller may be operable to perform a measurement using a sensor of the wearable article.

Advantageously, the controller may trigger the performance of measurements using the sensor if (only if) the wearer has been in the first motion state for more than a predetermined time period. This helps establish that the wearer is a settled, low, motion state such as sitting, standing or lying down and avoids performing measurements when the use is only temporarily in a low motion state such as during an intensive workout.

In response to the wearer being in the first motion state (e.g. for more than the first predetermined period of time), the controller may be operable to perform a plurality of measurements using the sensor. The measurements may be performed at a fixed or variable measurement/sampling rate. Example measurement rates include 100 Hz, 200 Hz, 300 Hz, 400 Hz, 500 Hz.

The controller may be operable to perform measurements using the sensor until an exit condition is reached.

The exit condition may be determined to be reached if more than a second predetermined time period has elapsed. This may mean that the controller performs measurements for a fixed period of time once it determines that the wearer is in the first motion state (e.g. for more than the first predetermined period of time). This enables sufficient measurements to be obtained to provide insights in relation to the health and/or wellbeing of the wearer such as those derived from the wearer's heart rate and/or heart rate variability. Ending the performance of measurements after the second predetermined time period has elapsed avoids performing unnecessary measurements which in turn reduces power and memory consumption.

The exit condition may be determined to be reached if the controller determines from contextual data that the wearer has transitioned from the first motion state to the second motion state. This may mean that the controller performs measurements until the wearer transitions to the second motion state. Ending the performance of measurements once the wearer transitions to the second measurement state avoids obtaining measurements with a high degree of motion artefact associated with wearer motion. This also avoids power and memory consumption issues associated with obtaining, storing and or communicating this noisy data.

The exit condition may be determined to be reached if the controller determines from contextual data that the wearer has been in the second motion state for more than a third predetermined time period. This may mean that the controller does not stop measurements if the wearer temporarily transitions to the second motion state for a short time period such as a time period of less than 5 seconds, or less than 1 second. This avoids exiting the measurement process as a result of temporary high motion movements by the wearer.

The exit condition may be determined to be reached if the controller determines that one or more of the measurements performed using the sensor do not satisfy a quality metric. One or more of the measurements performed using the sensor may be determined to not satisfy a quality metric if the maximum amplitude is less than a predetermined threshold and/or if the signal to noise ratio is below a predetermined threshold. The measurements may not satisfy a quality metric due to the presence of noise, for example, such as motion noise. This provides an additional mechanism for exiting the measurements where motion noise is present.

The exit condition may be determined to be reached if the controller receives a user input indicating that measurements should be stopped. That is, the user may send commands to the controller to exit the measurements. The commands may be send by a user electronic device in communication with the controller.

Any combination of the above exit conditions may be used.

In the first measurement mode, the controller may be operable to determine whether the wearable article is being worn, and in response to determining that the wearable article is being worn and the wearer is in the first motion state, perform the measurement using the sensor of the wearable article. In this way, the controller avoids performing the measurements if the wearable article is not being worn and thus not in a position to measure signals from the body surface. This may occur when the wearable article is held in a gym bag for example.

In the first measurement mode, the controller may be operable to determine whether the wearable article is being worn, and in response to determining that the wearable article is being worn and the wearer is in the first motion state, perform a plurality of measurements over a first predetermined time period.

In the first measurement mode, the controller may be operable to determine whether the wearable article is being worn, and in response to determining that the wearable article is being worn and the wearer has been in the first motion state for more than a first predetermined time period, perform the measurement using the sensor of the wearable article.

In response to determining that the wearable article is being worn, the controller may be operable to obtain the contextual data so as to determine whether the wearer of the wearable article is in the first motion state (e.g. for more than a first predetermined time period). Advantageously, contextual data may only be obtained once the controller determines that the wearable article is being worn. This can reduce power consumption as determining whether the wearable article is being worn is generally a less power intensive process than determining the motion state of the wearer.

In response to determining that the wearable article is being worn, the controller may be operable to transition from a first power mode to a second power mode that consumes more power than the first power mode. Advantageously, the controller may transition from a first, lower, power mode to a second, higher, power mode in response to determining that the wearable article is being worn. Prior to the controller determining that the wearable article is being worn, components of the wearable article such as the controller are operating in a first, low, power mode. This may mean that they are not supplied with power or only supplied with a minimal amount of power such as for refreshing an internal memory. This reduces unnecessary power consumption for the wearable article. Once the wearable is article is being worn, the controller wakes-up and obtains the contextual data and performs the other operations described above. This approach avoids unnecessary power consumption. This means that frequent charging or replacement of power sources for the wearable article is not required. Therefore, the present disclosure improves energy efficiency and reduces the environmental impact of the wearable article.

When in the second power mode, the controller may be operable to obtain the contextual data so as to determine whether the wearer of the wearable article is in the first motion state (e.g. for more than a first predetermined time period).

The controller determining whether the wearable article is being worn may comprise the controller determining whether the sensor is in contact with a body surface of the wearer. The controller may receive a signal indicating that the sensor is in contact with a body surface of the wearer. The signal may indicate a potential difference change brought about as a result of the sensor being placed in contact with the body surface.

The controller may be operable to enter the first measurement mode according to a predetermined schedule. For example, at predetermined times during a day, the controller may enter the first measurement mode and perform the steps outline above. This enables the health and/or wellbeing of the wearer to be monitored without necessarily requiring direction instruction from the user and while avoiding the power consumption and memory consumption associated with continual monitoring of the wearer.

The contextual data may comprise motion data for the wearer, and the controller may be operable to determine whether the wearer is in the first or second motion state from the motion data.

The contextual data may comprise location data for the wearer, and the controller may be operable to determine whether the wearer is in the first or second motion state from the location data.

The contextual data may comprise an input from a user indicating that the wearer is in the first motion state. The user may be different to or the same as the wearer. The input from the user may be received from an external device. The input from the user may be received via an electronics module that the controller is provided in.

Any combination of the contextual data described above may be used.

The controller may be operable to use the measurement performed in the first measurement mode to configure an algorithm which receives, as input, one or more measurements from the sensor. Advantageously, the controller is able to use the measurement as a baseline or reference measurement to configure an algorithm. As the measurement was obtained in the first motion state, the measurement is likely to be accurate and reliable.

The algorithm may comprise a peak detection algorithm arranged to detect peaks in a measurement signal received from the sensor, and wherein the controller is operable to configure a filtering or threshold parameter for the peak detection algorithm using the measurement performed in the first measurement mode. The threshold parameter may set the amplitude above which peaks are considered to be true peaks (e.g. R-peaks in an ECG signal) and below which peaks are considered to be due to noise. The filtering parameter may adjust a property of a low pass, high pass, bandpass or other form of filter used in removing artefacts from the signal prior to or after peak detection.

The controller may be operable to enter a second measurement mode. In the second measurement mode, the controller may be operable to repeatedly perform measurements using the sensor of the wearable article regardless of the motion state of the wearer. In the second measurement mode, the controller may be operable to perform measurements using the sensor at a first measurement rate independent of the motion state of the wearer. The second measurement mode may be desired when the controller is communicatively coupled to a wearable article designed to hold the sensors in close contact with the body surface and/or close to the heart of the wearer. Such a wearable article may be intended to be used while the wearer is exercising.

The controller may be operable to determine from contextual data whether to enter the first measurement mode or the second measurement mode. This enables the controller to intelligently switch between the first and second measurement mode based on the operating context of the controller.

The contextual data used to determine whether to enter the first measurement mode or the second measurement mode may comprise information about the wearable article the controller is communicatively coupled to.

The contextual data used to determine whether to enter the first measurement mode or the second measurement mode may comprise location information for the wearer.

The contextual data used to determine whether to enter the first measurement mode or the second measurement mode may comprise an input received from a user.

The contextual data used to determine whether to enter the first measurement mode or the second measurement mode may comprise information derived from one or more measurements performed using the sensor.

According to a second aspect of the disclosure, there is provided an electronics module comprising a controller as described above in relation to the first aspect of the disclosure.

The electronics module may be part of/removably coupled to the wearable article or may be separate from the wearable article and in wireless communication with the wearable article.

The sensor may be part of the wearable article. The sensor may be part of the electronics module. The sensor may be separate to the electronics module, such as by being incorporated into a fabric of the wearable article.

The electronics module may further comprise a contextual data unit. The contextual data unit may be a motion sensor, location data unit, or user input as described above in relation to the first aspect of the disclosure.

The electronics module may further comprise an interface arranged to communicate with the sensor of the wearable article.

The electronics module may further comprise a communicator arranged to communicate with an external device.

According to a third aspect of the disclosure, there is provided a system. The system comprises an electronics module according to the second aspect of the disclosure and a wearable article comprising a sensor.

The electronics module may be able to removably couple to the wearable article, or may be integrated with the wearable article or may be separate to the wearable article and in wireless communication with the wearable article.

The wearable article may be a garment.

According to a fourth aspect of the disclosure, there is provided a method performed by a controller for a wearable article. The method comprises: entering a first measurement mode, and in the first measurement mode: obtaining contextual data indicative of whether a wearer of the wearable article is in a first motion state or a second motion state representative of a higher degree of activity of the wearer than the first motion state; determining from the contextual data whether the wearer of the wearable article is in the first motion state; and in response to the wearer being in the first motion state, performing a measurement using a sensor of the wearable article.

In response to the wearer being in the first motion state, the method may comprise performing a plurality of measurements using the sensor of the wearable article over a first predetermined time period, and, if the wearer remains in the first motion state during the first predetermined time period, storing the measurements.

The method may comprise determining from the contextual data whether the wearer of the wearable article has been in the first motion state for more than a first predetermined time period; and in response to the wearer being in the first motion state for more than the first predetermined time period, performing a measurement using a sensor of the wearable article.

According to a fifth aspect of the disclosure, there is provided a controller for a wearable article. The controller is operable to obtain contextual data relating to the operational context of the wearable article; determine from the contextual data whether to enter a first measurement mode or a second measurement mode; and
enter the first measurement mode or the second measurement mode according to the determination. In the first measurement mode, the controller is operable to selectively perform measurements using a sensor of the wearable article according to the motion state of the wearer. In the second measurement mode, the controller is operable to perform measurements using the sensor at a first measurement rate independent of the motion state of the wearer.

Advantageously, the controller is able to intelligently switch between a first measurement mode that selectively performs measurements according to the motion state of the wearer and a second measurement mode that performs measurements independently of the motion state of the wearer. This enables the controller to operate in the most effective measurement mode for its determined operating context.

The controller may have any of the features described above in relation to the first aspect of the disclosure. The controller may be part of an electronics module such as an electronics module according to the second aspect of the disclosure.

According to a sixth aspect of the disclosure, there is provided a controller for a wearable article, the controller is operable to: obtain contextual data indicative of whether a wearer of the wearable article is in a first motion state or a second motion state representative of a higher degree of activity of the wearer than the first motion state; determine from the contextual data whether the wearer of the wearable article is in the first motion state; in response to the wearer being in the first motion state, perform a measurement using a sensor of the wearable article; and use the measurement to configure an algorithm which uses, as an input, the measurement performed using the sensor.

Advantageously, the controller is able to use the measurement as a baseline or reference measurement to configure an algorithm. As the measurement was obtained in the first motion state, the measurement is likely to be accurate and reliable.

The controller may have any of the features described above in relation to the first aspect of the disclosure. The controller may be part of an electronics module such as an electronics module according to the second aspect of the disclosure.

According to a seventh aspect of the disclosure, there is provided a system comprising: a controller according to the first aspect of the disclosure; and a wearable article comprising a sensor arranged such that, when worn, the sensor is displaced from a heart region of the wearer.

Here, “displaced from the heart region” means that when the wearable article is worn, the sensor is not located close to the chest region of the wearer and is instead spaced away from the chest region. This means that when measuring ECG signals, for example, the measured signals are weaker than if the sensors were located in the chest region. The sensors may be located around the waist, shoulder, arm, ankle, or wrist for example.

Because the amplitude of the measured signals are weaker than if the sensors were located close to the heart region of the wearer, the measured signals are more susceptible to noise particularly due to wearer motion. A high degree of motion can introduce motion artefacts into the measured signal which make analysis of signal challenging. For example, due to the presence of motion artefacts in the signal, it can be challenging if not possible to identify the true characteristic peaks in the signal (e.g. R-peaks) for use in determining the heart rate and heart rate variability of the wearer (amongst other measures). By (only) performing measurements when the wearer is in the first motion state, the controller is able to avoid recording noisy signals while still providing the desired measurement functions. This approach also avoids undesirable power and memory consumption associated with continual monitoring using the sensors.

According to an eighth aspect of the disclosure, there is provided a system comprising a controller according to the first aspect of the disclosure; and a wearable article comprising a band arranged to surround a circumference of the wearer and a sensor provided on the band.

The band may be a waist band, arm band, wrist band, or chest band. The chest band may form all or part of a bra underband.

According to a ninth aspect of the disclosure, there is provided a system comprising a controller according to the first aspect of the disclosure; and a wearable article comprising a waist band and a sensor provided on the waist band.

According to a tenth aspect of the disclosure, there is provided a system comprising: a controller according to the first aspect of the disclosure; and a wearable article comprising an arm band and a sensor provided on the arm band.

According to an eleventh aspect of the disclosure, there is provided a system comprising: a controller according to the first aspect of the disclosure; and a wearable article comprising a wrist band and a sensor provided on the wrist band.

According to a twelfth aspect of the disclosure, there is provided a system comprising: a wearable article comprising a sensor arranged to measure a signal from a body surface of the wearer, wherein, when worn, the sensor is arranged to move relative to the body surface of the wearer; and a controller according to the first aspect of the disclosure.

The sensor being arranged to move relative to the body surface of the wearer means that the sensor is not held tightly in contact with the body surface such as through compression applied by the wearable article or by a conductive adhesive gel layer. Motion of the sensor can introduce motion artefacts into the measurements. However, selectively performing measurements based on the motion state overcomes this problem while also reducing the power and memory consumption of the controller.

According to a thirteenth aspect of the disclosure, there is provided a system comprising: a wearable article comprising: a front area arranged to cover at least part of the front of the wearer, a back area arranged to cover at least part of the back of the wearer, at least one shoulder region connecting the front area to the back area, the at least one shoulder region comprising a sensor. The system further comprises a controller according to the first aspect of the disclosure.

Advantageously, the wearable article comprises a sensor provided in a shoulder region of the wearable article. Positioning the sensor in the shoulder region means that the weight of the wearable article pulls the sensor downwards into contact with the shoulder of the wearer when the wearable article is worn. This helps ensure effective contact between the sensor and the body surface (particularly in low motion states) without requiring that the wearable article applies compression. The controller selectively performs measurements based on the motion state of the wearer to enable effective measurements to be obtained while avoiding unnecessary power and memory consumption.

The wearable article may be a garment such as a top. Example tops include t-shirts, shirts and bras.

According to a fourteenth aspect of the disclosure, there is provided a wearable article comprising: a front area arranged to cover at least part of the front of the wearer; a back area arranged to cover at least part of the back of the wearer, the back area comprising an electronics module holder arranged to removably retain an electronics module; and at least one shoulder region connecting the front area to the back area, the at least one shoulder region comprising a sensor. The electronics module holder is arranged such that when the electronics module is retained by the electronics module holder, the electronics module is communicatively coupled to the sensor.

Advantageously, the wearable article comprises a sensor provided in a shoulder region of the wearable article. Positioning the sensor in the shoulder region means that the weight of the wearable article pulls the sensor downwards into contact with the shoulder of the wearer when the wearable article is worn. This helps ensure effective contact between the sensor and the body surface (particularly in low motion states) without requiring that the wearable article applies compression.

Moreover, the wearable article further comprises an electronics module holder positioned on the back area of the wearable article. Providing the electronics module holder on the back area, and particularly on the upper back area close to the neck of the wearer and between the collar bones is advantageous particularly when the wearable article is worn in high impact/combat sports. In this position, the electronics module is unlikely to affect the comfort of the wearer if they fall or experience another form of impact such as a tackle or a collision in a team sport activity.

The wearable article may further comprise a communication pathway (e.g. an electrically conductive pathway) that extends from the sensor to the electronics module holder such that the electronics module holder is communicatively coupled to the sensor. The electronics module holder may be arranged such that when the electronics module is retained by the electronics module holder, the electronics module is communicatively coupled to the sensor via the communication pathway.

A system comprising the wearable article of the thirteenth aspect of the disclosure and an electronics module according to the second aspect of the disclosure is also provided.

According to a fifteenth aspect of the disclosure, there is provided a wearable article comprising: a front area arranged to cover at least part of the front of the wearer; a back area arranged to cover at least part of the back of the wearer, a pair of shoulder regions connecting the front area to the back area, at least one of the shoulder regions comprising an electronics module holder arranged to removably retain an electronics module; and a sensor. The electronics module holder is arranged such that when the electronics module is retained by the electronics module holder, the electronics module is communicatively coupled to the sensor.

Advantageously, the wearable article comprises an electronics module holder positioned on the shoulder region of the wearable article. Providing the electronics module holder on the shoulder region is advantageous particularly when the wearable article is worn in high impact/combat sports. In this position, the electronics module is unlikely to affect the comfort of the wearer if they fall or experience another form of impact such as a tackle or a collision in a team sport activity.

The wearable article may further comprise a communication pathway (e.g. an electrically conductive pathway) that extends from the sensor to the electronics module holder such that the electronics module holder is communicatively coupled to the sensor. The electronics module holder may be arranged such that when the electronics module is retained by the electronics module holder, the electronics module is communicatively coupled to the sensor via the communication pathway.

A system comprising the wearable article of the fourteenth aspect of the disclosure and an electronics module according to the second aspect of the disclosure is also provided.

According to a sixteenth aspect of the disclosure, there is provided a wearable article comprising: a front area arranged to cover at least part of the front of the wearer; a back area arranged to cover at least part of the back of the wearer; a pair of shoulder regions connecting the front area to the back area; a collar region provided between the pair of shoulder regions and defining a neck opening, the collar region comprising an electronics module holder arranged to removably retain an electronics module; and a sensor. The electronics module holder is arranged such that when the electronics module is retained by the electronics module holder, the electronics module is communicatively coupled to the sensor.

Advantageously, the wearable article comprises an electronics module holder positioned on the collar region of the wearable article. Providing the electronics module holder on the collar region is advantageous particularly when the wearable article is worn in high impact/combat sports. In this position, the electronics module is unlikely to affect the comfort of the wearer if they fall or experience another form of impact such as a tackle or a collision in a team sport activity.

The wearable article may further comprise a communication pathway (e.g. an electrically conductive pathway) that extends from the sensor to the electronics module holder such that the electronics module holder is communicatively coupled to the sensor. The electronics module holder may be arranged such that when the electronics module is retained by the electronics module holder, the electronics module is communicatively coupled to the sensor via the communication pathway.

A system comprising the wearable article of the fifteenth aspect of the disclosure and an electronics module according to the second aspect of the disclosure is also provided.

According to a seventeenth aspect of the disclosure, there is provided a shirt collar assembly comprising: a collar stand; a shirt collar attached at an upper portion of the collar stand. An inner side of the shirt collar faces an outer side of the collar stand when the shirt collar is in a folded down position relative to the collar stand. At least one of the inner side of the shirt collar and the outer side of the collar stand comprises an electronics module holder arranged to receive an electronics module, wherein the electronics module holder comprises a communication interface arranged to communicatively couple with the electronics module when positioned in the electronics module holder.

Advantageously, positioning the electronics module holder on the shirt collar assembly is convenient and easy to use for the wearer. It is easy to insert and remove the electronics module from the electronics module holder when the wearable article is being worn.

The communication interface may comprise a conductive interface.

The communication interface may comprise a wireless communication interface.

The electronics module holder may be provided on the inner side of the shirt collar.

The electronics module holder may be provided on the outer side of the collar stand.

The electronics module holder may comprise a pocket.

There is also provided a wearable article comprising the shirt collar assembly of the sixteenth aspect of the disclosure.

The wearable article may further comprise a sensor communicatively coupled to the communication interface by a communication pathway.

The wearable article may comprise a front area arranged to cover at least part of the front of the wearer; a back area arranged to cover at least part of the back of the wearer; and a pair of shoulder regions connecting the front area to the back area, wherein the shirt collar assembly is provided between the pair of shoulder regions.

A system comprising the wearable article and an electronics module according to the second aspect of the disclosure is also provided.

According to an eighteenth aspect of the disclosure, there is provided a system comprising an electronics module and a wearable article.

The electronics module comprises a controller and at least one electrical contact communicatively coupled to the controller. The controller is operable to receive biosignals from the at least one electrical contact

The wearable article comprises a pocket sized to receive the electronics module. An internal layer of the pocket comprises at least one opening sized to receive the at least one electrical contact. When the electronics module is positioned in the pocket, the at least one electrical contact is arranged to extend through the at least one opening so as to contact a skin surface of the wearer when the wearable article is worn.

The at least one electrical contact therefore forms a sensor for the electronics module (e.g. an electrode) and is able to provide biosignals to the controller without coupling to a separate sensor in the wearable article.

The at least one electrical contact may comprise a plurality of electrical contacts.

The at least one opening may comprise a plurality of openings. Each of the plurality of openings may be sized to receive one of a plurality of electrical contacts of the electronics module.

The at least one opening may comprise a single opening. The single opening may be sized to receive a plurality of electrical contacts of the electronics module.

The at least one opening may have a complementary shape to the at least one electrical contact.

The electronics module may be the electronics module of the second aspect of the disclosure.

According to an nineteenth aspect of the disclosure, there is provided a system comprising an electronics module according to the second aspect of the disclosure and a wearable article.

The electronics module comprises a controller and at least one electrical contact communicatively coupled to the processor. The controller is operable to receive biosignals from the at least one electrical contact

The wearable article comprises at least one opening sized to receive the at least one electrical contact. When the electronics module is positioned on the wearable article, the at least one electrical contact is arranged to extend through the at least one opening so as to contact a skin surface of the wearer when the wearable article is worn.

The at least one electrical contact therefore forms a sensor for the electronics module (e.g. an electrode) and is able to provide biosignals to the controller without coupling to a separate sensor in the wearable article.

According to a twentieth aspect of the disclosure, there is provided system comprising an electronics module and a wearable article, the electronics module comprises a controller and at least one sensing unit communicatively coupled to the controller, the controller is operable to receive signals (e.g. bio/physiological signals) from the at least one sensing unit, the wearable article comprises at least one opening sized to receive the at least one sensing unit, wherein when the electronics module is positioned on the wearable article, the at least one sensing unit is arranged to extend through the at least one opening so as to contact a skin surface of the wearer when the wearable article is worn, wherein the controller is operable to enter a first measurement mode, wherein in the first measurement mode, the controller is operable to: obtain contextual data indicative of whether a wearer of the wearable article is in a first motion state or a second motion state representative of a higher degree of activity of the wearer than the first motion state; determine from the contextual data whether the wearer of the wearable article is in the first motion state; and in response to the wearer being in the first motion state, perform a measurement using the sensing unit.

The sensing unit may be an electrical contact, such as for ECG measurements. The sensing unit may be a sensor such as an optical sensor. An optical sensor may be used for PPG measurements.

According to a twenty first aspect of the disclosure, there is provided a shirt collar assembly comprising: a collar stand; a shirt collar attached at an upper portion of the collar stand, wherein an inner side of the shirt collar faces an outer side of the collar stand when the shirt collar is in a folded down position relative to the collar stand, and wherein at least one of the inner side of the shirt collar and the outer side of the collar stand comprises an electronics module holder arranged to receive an electronics module, wherein the collar stand or electronics module holder is constructed such that when an electronics module is positioned in the electronics module holder, a sensor of the electronics module has line of sight with a skin surface of the wearer.

The electronics module holder may be provided on an inner side of the collar stand. The electronics module holder may be constructed such that when the electronics module is positioned in the electronics module holder, the sensor of the electronics module has line of sight with the skin surface of the wearer.

The electronics module holder may be provided on the outer side of the collar stand.

The collar stand may be constructed such that when the electronics module is positioned in the electronics module holder, the sensor of the electronics module has line of sight with the skin surface of the wearer.

The electronics module holder may be a pocket.

The collar stand or electronics module holder may comprise an opening or window, and wherein the sensor of the electronics module, when positioned in the electronics module holder is aligned with the opening or window.

According to a twenty second aspect of the disclosure, there is provided a system comprising an electronics module and a wearable article, the electronics module comprises a controller and a sensor communicatively coupled to the controller, the wearable article comprises at least one opening or window, wherein when the electronics module is positioned on the wearable article, the sensor has line of sight with a skin surface of a wearer through the opening or window, wherein the controller is operable to enter a first measurement mode, wherein in the first measurement mode, the controller is operable to: obtain contextual data indicative of whether a wearer of the wearable article is in a first motion state or a second motion state representative of a higher degree of activity of the wearer than the first motion state; determine from the contextual data whether the wearer of the wearable article is in the first motion state; and in response to the wearer being in the first motion state, perform a measurement using the sensor.

According to a twenty third aspect of the disclosure, there is provided a wearable article comprising: a front area arranged to cover at least part of the front of the wearer; a back area arranged to cover at least part of the back of the wearer; a pair of shoulder regions connecting the front area to the back area; a collar region provided between the pair of shoulder regions and defining a neck opening, the collar region comprising an electronics module holder arranged to removably retain an electronics module. The collar region is constructed such that when the electronics module is retained by the electronics module holder, a sensor of the electronics module has line of sight with a skin surface of the wearer.

The collar region may comprise an opening or window. The sensor of the electronics module, when positioned in the electronics module, is aligned with the opening or window in the collar region.

According to a twenty fourth aspect of the disclosure, there is provided a wearable article comprising: a front area arranged to cover at least part of the front of the wearer; a back area arranged to cover at least part of the back of the wearer, a pair of shoulder regions connecting the front area to the back area, at least one of the shoulder regions comprising an electronics module holder arranged to removably retain an electronics module. The shoulder region is constructed such that when the electronics module is retained by the electronics module holder, a sensor of the electronics module has line of sight with a skin surface of the wearer.

The shoulder region may comprise an opening or window, and wherein the sensor of the electronics module, when positioned in the electronics module, is aligned with the opening or window in the collar region.

### BRIEF DESCRIPTION OF THE DRAWINGS

Examples of the present disclosure will now be described with reference to the accompanying drawings, in which:

FIG. 1 shows a schematic diagram for an example system according to aspects of the present disclosure;

FIG. 2 shows a schematic diagram for an example electronics module according to aspects of the present disclosure;

FIG. 3 shows a schematic diagram for another example electronics module according to aspects of the present disclosure;

FIG. 4 shows a schematic diagram for an example analogue-to-digital converter used in the example electronics module of FIGS. 2 and 3 according to aspects of the present disclosure;

FIG. 5 shows a schematic diagram of a user electronic device according to aspects of the present disclosure; and

FIGS. 6 and 7 show an example plots of ECG traces;

FIG. 8 shows a flow diagram for an example method according to aspects of the present disclosure;

FIG. 9 shows a flow diagram for another example method according to aspects of the present disclosure;

FIG. 10 shows a flow diagram for another example method according to aspects of the present disclosure;

FIG. 11 shows a flow diagram for another example method according to aspects of the present disclosure;

FIG. 12 shows a flow diagram for another example method according to aspects of the present disclosure;

FIG. 13 shows an example wearable article according to aspects of the present disclosure;

FIGS. 14-15 show front and back views of an example wearable article according to aspects of the present disclosure;

FIG. 16 shows the back view of another example wearable article according to aspects of the present disclosure;

FIG. 17 shows the back view of another example wearable article according to aspects of the present disclosure;

FIG. 18 shows the front view of another example wearable article according to aspects of the present disclosure;

FIG. 19 shows the front view of another example wearable article according to aspects of the present disclosure;

FIGS. 20 and 21 show views of an example electronics module according to aspects of the present disclosure;

FIG. 22 shows a detail of the wearable article in FIG. 19; and

FIGS. 23 to 25 show the electronics module of FIGS. 20 and 21 positioned on the wearable article in FIG. 22.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings but are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise.

“Wearable article” as referred to throughout the present disclosure may refer to any form of article which may be worn by a user such as a smart watch, necklace, garment, bracelet, or glasses. The wearable article may be a textile article. The wearable article may be a garment. The garment may refer to an item of clothing or apparel. The garment may be a top. The top may be a shirt, t-shirt, blouse, sweater, jacket/coat, or vest. The garment may be a dress, garment brassiere, shorts, pants, arm or leg sleeve, vest, jacket/coat, glove, armband, underwear, headband, hat/cap, collar, wristband, armband, chestband, waistband, stocking, sock, or shoe, athletic clothing, personal protective equipment, including hard hats, swimwear, wetsuit or dry suit.

The term “wearer” includes a user who is wearing, or otherwise holding, the wearable article.

The type of wearable garment may dictate the type of biosignals to be detected. For example, a hat or cap may be used to detect electroencephalogram or magnetoencephalogram signals.

The wearable article/garment may be constructed from a woven or a non-woven material. The wearable article/garment may be constructed from natural fibres, synthetic fibres, or a natural fibre blended with one or more other materials which can be natural or synthetic. The yarn may be cotton. The cotton may be blended with polyester and/or viscose and/or polyamide according to the application. Silk may also be used as the natural fibre. Cellulose, wool, hemp and jute are also natural fibres that may be used in the wearable article/garment. Polyester, polycotton, nylon and viscose are synthetic fibres that may be used in the wearable article/garment.

The garment may be a tight-fitting garment or a loose-fitting (e.g. freeform garment). A tight-fitting garment helps ensure that the sensor devices of the garment are held in contact with or in the proximity of a skin surface of the wearer. The tight-fitting garment may be a compression garment. The tight-fitting garment may be an athletic garment such as an elastomeric athletic garment. A loose-fitting garment is generally more comfortable to wear over extending time periods and during sleep.

The garment has sensing units provided on an inside surface which are held in close proximity to a skin surface of a wearer wearing the garment. This enables the sensing units to measure biosignals for the wearer wearing the garment.

The sensing units may be arranged to measure one or more biosignals of a wearer wearing the garment.

“Biosignal” as referred to throughout the present disclosure may refer to signals from living beings that can be continually measured or monitored. Biosignals may be electrical or non-electrical signals. Signal variations can be time variant or spatially variant.

Sensing units may be used for measuring one or a combination of bioelectrical, bioimpedance, biochemical, biomechanical, bioacoustics, biooptical or biothermal signals of the wearer 600. The sensing units may be incorporated into the wearable article, an electronics module coupled to or forming part of the wearable article, or may be shared between the electronics module and the wearable article. For example, the wearable article may comprise sensors (e.g. sensing electrodes) while the electronics module may comprise the processing logic for the sensing electrodes. The processing logic will review the signals from the sensors and perform operations such as filtering and analogue-to-digital conversion on the signals. The bioelectrical measurements include electrocardiograms (ECG), electrogastrograms (EGG), electroencephalograms (EEG), and electromyography (EMG). The bioimpedance measurements include plethysmography (e.g., for respiration), body composition (e.g., hydration, fat, etc.), and electroimpedance tomography (EIT). The biomagnetic measurements include magnetoneurograms (MNG), magnetoencephalography (MEG), magnetogastrogram (MGG), magnetocardiogram (MCG). The biochemical measurements include glucose/lactose measurements which may be performed using chemical analysis of the wearer 600's sweat. The biomechanical measurements include blood pressure. The bioacoustics measurements include phonocardiograms (PCG). The biooptical measurements include photoplethysmography (PPG) and orthopantomograms (OPG). The biothermal measurements include skin temperature and core body temperature measurements.

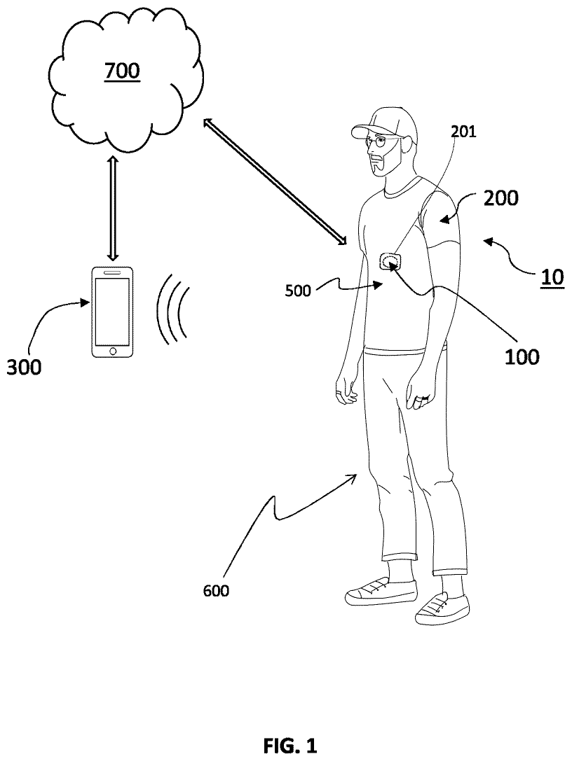

Referring to FIGS. 1 to 6, there is shown an example system 10 according to aspects of the present disclosure. The system 10 comprises an electronics module 100, a wearable article in the form of a garment 200, and a user electronic device 300. The garment 200 is worn by a user who in this embodiment is the wearer 600 of the garment 200.

The electronics module 100 is arranged to integrate with sensing units 400 incorporated into the garment 200 to obtain signals from the sensing unit 400.

The electronics module 100 and the wearable article 200 and including the sensing units 400 comprise a wearable assembly 500.

The sensing units 400 comprise one or more sensors 209, 211 with associated conductors 203, 207 and other components and circuitry. The electronics module 100 is further arranged to wirelessly communicate data to the user electronic device 300. Various protocols enable wireless communication between the electronics module 100 and the user electronic device 300. Example communication protocols include Bluetooth®, Bluetooth® Low Energy, and near-field communication (NFC).

The garment 200 has an electronics module holder in the form of a pocket 201. The pocket 201 is sized to receive the electronics module 100. When disposed in the pocket 201, the electronics module 100 is arranged to receive sensor data from the sensing units 400. The electronics module 100 is therefore removable from the garment 200.

The present disclosure is not limited to electronics module holders in the form pockets.

The electronics module 100 may be configured to be releasably mechanically coupled to the garment 200. The mechanical coupling of the electronic module 100 to the garment 200 may be provided by a mechanical interface such as a clip, a plug and socket arrangement, etc. The mechanical coupling or mechanical interface may be configured to maintain the electronic module 100 in a particular orientation with respect to the garment 200 when the electronic module 100 is coupled to the garment 200. This may be beneficial in ensuring that the electronic module 100 is securely held in place with respect to the garment 200 and/or that any electronic coupling of the electronic module 100 and the garment 200 (or a component of the garment 200) can be optimized. The mechanical coupling may be maintained using friction or using a positively engaging mechanism, for example.

Beneficially, the removable electronic module 100 may contain all the components required for data transmission and processing such that the garment 200 only comprises the sensing units 400 e.g. the sensors 209, 211 and communication pathways 203, 207. In this way, manufacture of the garment 200 may be simplified. In addition, it may be easier to clean a garment 200 which has fewer electronic components attached thereto or incorporated therein. Furthermore, the removable electronic module 100 may be easier to maintain and/or troubleshoot than embedded electronics. The electronic module 100 may comprise flexible electronics such as a flexible printed circuit (FPC).

The electronic module 100 may be configured to be electrically coupled to the garment 200.

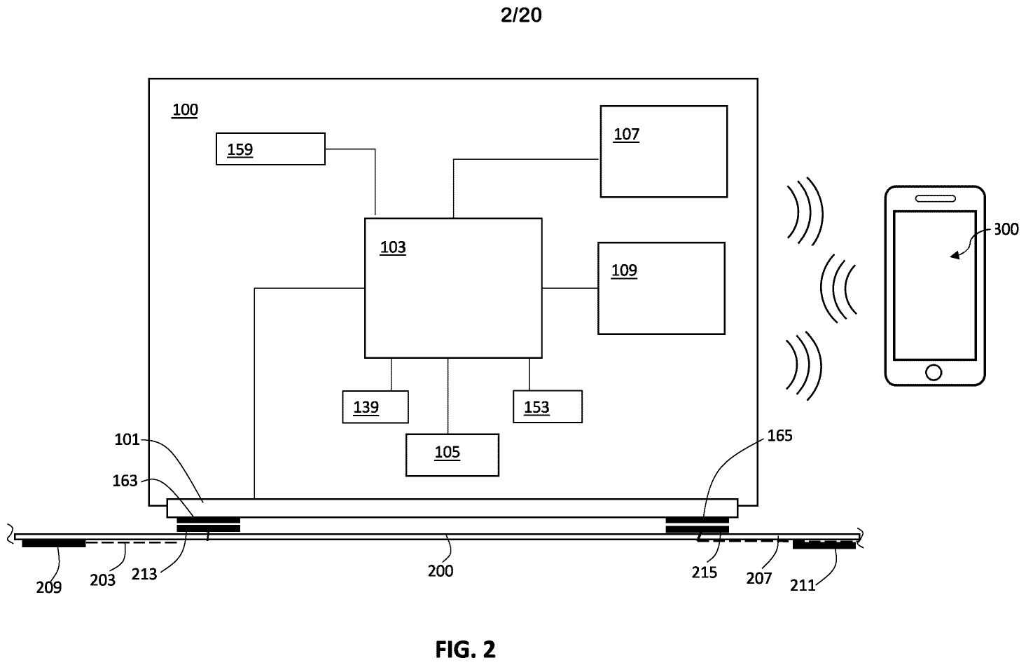

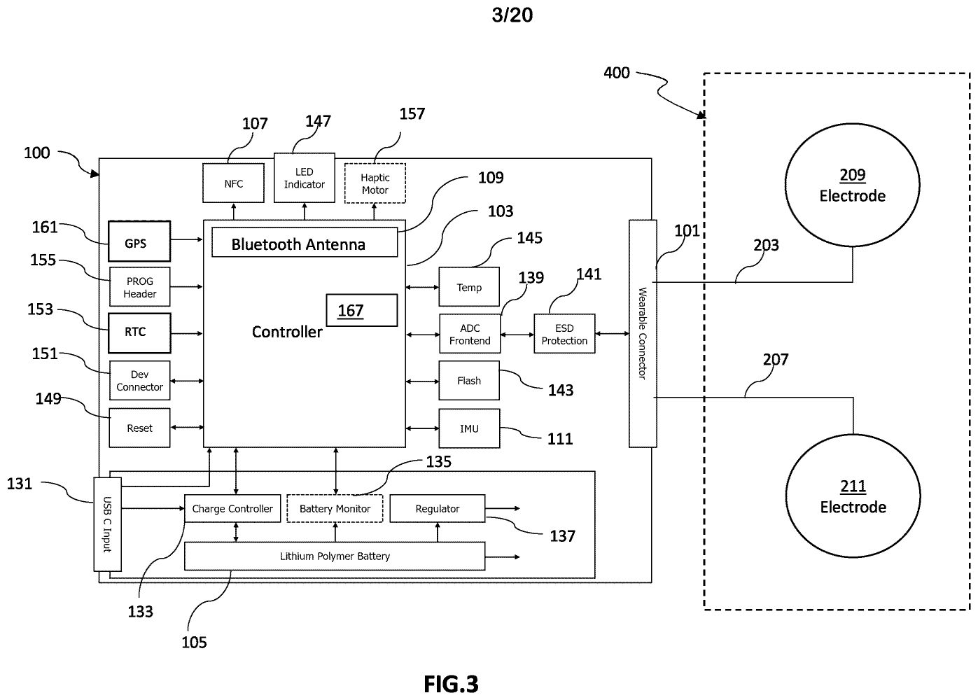

Referring to FIG. 2, there is shown a schematic diagram of an example of the electronics module 100. A more detailed block diagram of the electronics components of electronics module 100 and garment are shown in FIG. 3. It will be appreciated that not all of the components shown in FIGS. 2 and 3 are required and additional component may also be provided.

The electronics module 100 comprises an interface 101, a controller 103, a power source 105, and one or more communication devices which, in the exemplar embodiment comprises a first antenna 107, a second antenna 109 and a wireless communicator 159.

The electronics module 100 also includes additional peripheral devices that are used to perform specific functions as will be described in further detail herein.

The interface 101 is arranged to communicatively couple with the sensing unit 400 of the garment 200. The sensing unit 400 comprises—in this example—the two sensors 209, 211 coupled to respective first and second electrically conductive pathways 203, 207, each with respective termination points 213, 215 (also referred to as connection regions 213, 215). The interface 101 receives signals from the sensors 209, 211. The controller 103 is communicatively coupled to the interface 101 and is arranged to receive the signals from the interface 101 for further processing.

The interface 101 of the embodiment described herein comprises first and second contacts 163, 165 which are arranged to be communicatively coupled to the termination points 213, 215 the respective first and second electrically conductive pathways 203, 207. The coupling between the termination points 213, 215 and the respective first and second contacts 163, 165 may be conductive or a wireless (e.g. inductive) communication coupling.

In this example the sensors 209, 211 are electrodes used to measure electropotential signals such as electrocardiogram (ECG) signals, although the sensors 209, 211 could be configured to measure other biosignal types as also discussed above. For example, the electrodes may also be used to perform bioimpedance measurements such as for impedance plethysmography.

In this embodiment, the sensors 209, 211 are configured for so-called dry connection to the wearer's skin to measure ECG signals. In a dry connection configuration, an adhesive gel layer is not provided to couple the sensors 209, 211 to the skin surface. Instead, the sensors 209, 211 are not held fixedly in place and may be able to move relative to the skin surface depending on the tightness of the wearable article 100 and the positioning of the sensors 209, 211 on the wearable article 100.

The power source 105 may comprise one or a plurality of power sources. The power source 105 may be a battery. The battery may be a rechargeable battery. The battery may be a rechargeable battery adapted to be charged wirelessly such as by inductive charging. The power source 105 may comprise an energy harvesting device. The energy harvesting device may be configured to generate electric power signals in response to kinetic events such as kinetic events performed by the wearer 600 of the garment 200. The kinetic event could include walking, running, exercising or respiration of the wearer 600. The energy harvesting material may comprise a piezoelectric material which generates electricity in response to mechanical deformation of the converter. The energy harvesting device may harvest energy from body heat of the wearer 600 of the garment. The energy harvesting device may be a thermoelectric energy harvesting device. The power source 105 may be a super capacitor, or an energy cell.

The first antenna 107 is arranged to communicatively couple with the user electronic device 300 using a first communication protocol. In the example described herein, the first antenna 107 is a passive tag such as a passive Radio Frequency Identification (RFID) tag or Near Field Communication (NFC) tag. These tags comprise a communication module as well as a memory which stores the information, and a radio chip. The user electronic device 300 is powered to induce a magnetic field in an antenna of the user electronic device 300. When the user electronic device 300 is placed in the magnetic field of the communication module antenna 107, the user electronic device 300 induces current in the communication module antenna 107. This induced current is used to retrieve the information from the memory of the tag and transmit the same back to the user electronic device 300. The controller 103 is arranged to energize the first antenna 107 to transmit information.

In an example operation, the user electronic device 300 is brought into proximity with the electronics module 100. In response to this, the electronics module 100 is configured to energize the first antenna 107 to transmit information to the user electronic device 300 over the first wireless communication protocol. Beneficially, this means that the act of the user electronic device 300 approaching the electronics module 100 energizes the first antenna 107 to transmit the information to the user electronic device 300.

The information may comprise a unique identifier for the electronics module 100. The unique identifier for the electronics module 100 may be an address for the electronics module 100 such as a MAC address or Bluetooth® address.

The information may comprise authentication information used to facilitate the pairing between the electronics module 100 and the user electronic device 300 over the second wireless communication protocol. This means that the transmitted information is used as part of an out of band (OOB) pairing process.

The information may comprise application information which may be used by the user electronic device 300 to start an application on the user electronic device 300 or configure an application running on the user electronic device 300. The application may be started on the user electronic device 300 automatically (e.g. without wearer 600 input). Alternatively, the application information may cause the user electronic device 300 to prompt the wearer 600 to start the application on the user electronic device 300. The information may comprise a uniform resource identifier such as a uniform resource location to be accessed by the user electronic device 300, or text to be displayed on the user electronic device for example. It will be appreciated that the same electronics module 100 can transmit any of the above example information either alone or in combination. The electronics module 100 may transmit different types of information depending on the current operational state of the electronics module 100 and based on information it receives from other devices such as the user electronic device 300.

The second antenna 109 is arranged to communicatively couple with the user electronic device 300 over a second wireless communication protocol. The second wireless communication protocol may be a Bluetooth® protocol, Bluetooth® 5 or a Bluetooth® Low Energy protocol but is not limited to any particular communication protocol. In the present embodiment, the second antenna 109 is integrated into controller 103. The second antenna 109 enables communication between the user electronic device 300 and the controller 103 for configuration and set up of the controller 103 and the peripheral devices as may be required.

Configuration of the controller 103 and peripheral devices utilises the Bluetooth® protocol.

Other wireless communication protocols can also be used, such as used for communication over: a wireless wide area network (WWAN), a wireless metro area network (WMAN), a wireless local area network (WLAN), a wireless personal area network (WPAN), Bluetooth® Low Energy, Bluetooth® Mesh, Thread, Zigbee, IEEE 802.15.4, Ant, a Global Navigation Satellite System (GNSS), a cellular communication network, or any other electromagnetic RF communication protocol. The cellular communication network may be a fourth generation (4G) LTE, LTE Advanced (LTE-A), LTE Cat-M1, LTE Cat-M2, NB-IOT, fifth generation (5G), sixth generation (6G), and/or any other present or future developed cellular wireless network.

The electronics module 100 includes a clock unit in the form of a real time clock (RTC) 153 coupled to the controller 103 and, for example, to be used for data logging, clock building, time stamping, timers, and alarms. As an example, the RTC 153 is driven by a low frequency clock source or crystal operated at 32.768 Hz.

The electronics module 100 also includes a location device 161 such as a GNSS (Global Navigation Satellite System) device which is arranged to provide location and position data for applications as required. In particular, the location device 161 provides geographical location data at least to a nation state level. Any device suitable for providing location, navigation or for tracking the position could be utilised. The GNSS device may include Global Positioning System (GPS), BeiDou Navigation Satellite System (BDS) and the Galileo system devices.

The power source 105 in this example is a lithium polymer battery 105. The battery 105 is rechargeable and charged via a USB C input 131 of the electronics module 100. Of course, the present disclosure is not limited to recharging via USB and instead other forms of charging such as inductive of far field wireless charging are within the scope of the present disclosure. Additional battery management functionality is provided in terms of a charge controller 133, battery monitor 135 and regulator 147. These components may be provided through use of a 30 dedicated power management integrated circuit (PMIC).

The USB C input 131 is also coupled to the controller 131 to enable direct communication between the controller 103 and an external device if required.

The controller 103 is communicatively connected to a battery monitor 135 so that that the controller 103 may obtain information about the state of charge of the battery 105.

The controller 103 has an internal memory 167 and is also communicatively connected to an external memory 143 which in this example is a NAND Flash memory. The memory 143 is used to for the storage of data when no wireless connection is available between the electronics module 100 and a user electronic device 300. The memory 143 may have a storage capacity of at least 1 GB and preferably at least 2 GB.

The electronics module 100 also comprises a temperature sensor 145 and a light emitting diode 147 for conveying status information about the electronics module and/or the wearer of the electronics module 100. The electronic module 100 also comprises conventional electronics components including a power-on-reset generator 149, a development connector 151, the real time clock 153 and a PROG header 155.

Additionally, the electronics module 100 may comprise a haptic feedback unit 157 for providing a haptic (vibrational) feedback to the wearer 600.

The wireless communicator 159 may provide wireless communication capabilities for the garment 200 and enables the garment to communicate via one or more wireless communication protocols to a remote server 700. Wireless communications may include: a wireless wide area network (WWAN), a wireless metro area network (WMAN), a wireless local area network (WLAN), a wireless personal area network (WPAN), Bluetooth® Low Energy, Bluetooth® Mesh, Bluetooth® 5, Thread, Zigbee, IEEE 802.15.4, Ant, a near field communication (NFC), a Global Navigation Satellite System (GNSS), a cellular communication network, or any other electromagnetic RF communication protocol. The cellular communication network may be a fourth generation (4G) LTE, LTE Advanced (LTE-A), LTE Cat-M1, LTE Cat-M2, NB-IOT, fifth generation (5G), sixth generation (6G), and/or any other present or future developed cellular wireless network.

The wireless communicator 159 may be an alternative, or in addition to, the first and second antennas 107, 109.

The electronics module 100 may additionally comprise a Universal Integrated Circuit Card (UICC) that enables the garment to access services provided by a mobile network operator (MNO) or virtual mobile network operator (VMNO). The UICC may include at least a read-only memory (ROM) configured to store an MNO or VMNO profile that the garment can utilize to register and interact with an MNO or VMNO. The UICC may be in the form of a Subscriber Identity Module (SIM) card. The electronics module 100 may have a receiving section arranged to receive the SIM card. In other examples, the UICC is embedded directly into a controller of the electronics module 100. That is, the UICC may be an electronic/embedded UICC (eUICC). A eUICC is beneficial as it removes the need to store a number of MNO profiles, i.e. electronic Subscriber Identity Modules (eSIMs). Moreover, eSIMs can be remotely provisioned to garments. The electronics module 100 may comprise a secure element that represents an 35 embedded Universal Integrated Circuit Card (eUICC). In the present disclosure, the electronics module may also be referred to as an electronics device or unit. These terms may be used interchangeably.

The controller 103 is connected to the interface 101 via an analog-to-digital converter (ADC) front end 139 and optionally an electrostatic discharge (ESD) protection circuit 141.

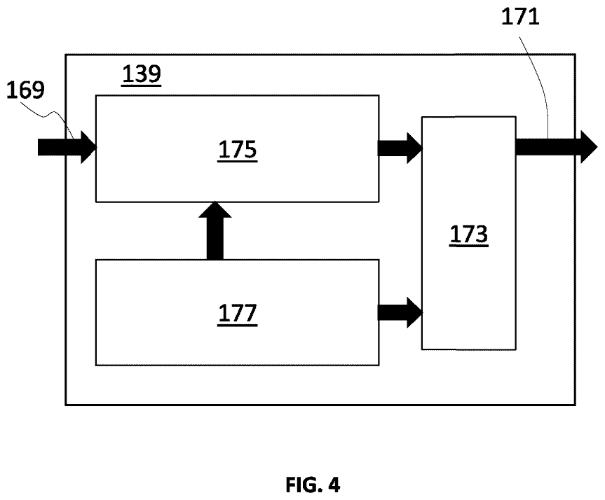

FIG. 4 is a schematic illustration of the component circuitry for the ADC front end 139.

In the example described herein, the ADC front end 139 is an integrated circuit (IC) chip which converts the raw analogue biosignal received from the sensors 209, 211 into a digital signal for further processing by the controller 103. ADC IC chips are known, and any suitable one can be utilised to provide this functionality. ADC IC chips for ECG applications include, for example, the MAX30003 chip produced by Maxim Integrated Products Inc.

The ADC front end 139 includes an input 169 and an output 171.

Raw biosignals from the electrodes 209, 211 are input to the ADC front end 139, where received signals are processed in an ECG channel 175 and subject to appropriate filtering through high pass and low pass filters for static discharge and interference reduction as well as for reducing bandwidth prior to conversion to digital signals. The reduction in bandwidth is important to remove or reduce motion artefacts that give rise to noise in the signal due to movement of the sensors 209, 211.

The output digital signals may be decimated to reduce the sampling rate prior to being passed to a serial programmable interface (SPI) 173 of the ADC front end 139.

ADC front end IC chips suitable for ECG applications may be configured to determine information from the input biosignals such as heart rate and the QRS complex and including the R-R interval of the QRS complex. Support circuitry 177 provides base voltages for the ECG channel 175.

The determining of the QRS complex can be implemented for example using the known Pan Tomkins algorithm as described in Pan, Jiapu; Tompkins, Willis J. (March 1985). “A Real-Time QRS Detection Algorithm”. IEEE Transactions on Biomedical Engineering. BME-32 (3): 230-236.

Signals are output to the controller 103 via the SPI 173.

The controller 103 can also be configured to apply digital signal processing (DSP) to the digital signal from the ADC front end 139.

The DSP may include noise filtering additional to that carried out in the ADC front end 139 and may also include additional processing to determine further information about the signal from the ADC front end 139.

The controller 103 is configured to send the biosignals to the user electronic device 300 using either of the first antenna 107, second antenna 109, or wireless communicator 159.

In some examples, the input unit—such as a proximity sensor or motion sensor—is arranged to detect a displacement of the electronics module 100. These displacements of the electronics module 100 may be caused by the object being tapped against the electronics module 100 or by the wearer 600 of the electronics module 100 being in motion, for example walking or running, or simply getting up from a recumbent position.

In the exemplar embodiment described herein, motion detection is provided by the IMU 111 which may comprise an accelerometer and optionally one or both of a gyroscope and a magnetometer. A gyroscope/magnetometer is not required in all examples, and instead only an accelerometer may be provided, or a gyroscope/magnetometer may be present but put into a low power state.

The input unit could be an AI system, machine or engine.

The IMU 111 can therefore be used to detect can detect orientation and gestures with event-detection interrupts enabling motion tracking and contextual awareness. It has recognition of free-fall events, tap and double-tap sensing, activity or inactivity, stationary/motion detection, and wakeup events in addition to 6D orientation. A single tap, for example, can be used enable toggling through various modes or waking the electronics module 100 from a low power mode.

Known examples of IMUs that can be used for this application include the ST LSM6DSOX manufactured by STMicroelectronics. This IMU a system-in-package IMU featuring a 3D digital accelerometer and a 3D digital gyroscope.

Another example of a known IMU suitable for this application is the LSM6DSO also be STMicroelectronics.

The IMU 111 can include machine learning functionality, for example as provided in the ST LSM6DSOX. The machine learning functionality is implemented in a machine learning core (MLC). The machine earning processing capability uses decision-tree logic. The MLC is an embedded feature of the IMU 111 and comprises a set of configurable parameters and decision trees. As is understood in the art, decision tree is a mathematical tool composed of a series of configurable nodes. Each node is characterized by an “if-then-else” condition, where an input signal (represented by statistical parameters calculated from the sensor data) is evaluated against a threshold.

Decision trees are stored and generate results in the dedicated output registers. The results of the decision tree can be read from the application processor at any time. Furthermore, there is the possibility to generate an interrupt for every change in the result in the decision tree, which is beneficial in maintaining low-power consumption.

Decision trees can be generated using a known machine learning tool such as Waikato Environment for Knowledge Analysis software (Weka) developed by the University of Waikato or using MATLAB® or Python™.

In an example operation, the wearer 600 has positioned the electronics module 100 within the pocket 201 (FIG. 1) of the garment 200 and is wearing the garment 200. The wearer 600 taps their hand or mobile phone 300 against the pocket 201 and this tap event is detected by the input unit, which in this exemplar embodiment is the IMU 111. The IMU 111 sends a signal to the controller 103 to wake-up the controller 103 from the low power mode.

A processor of the IMU 111 may perform processing tasks to classify different types of detected motion. The processor of the IMU 111 may use the machine-learning functions so as to perform this classification. Performing the processing operations on the IMU 111 rather than the controller 103 is beneficial as it reduces power consumption and leaves the controller 103 free to perform other tasks. In addition, it allows for motion events to be detected even when the controller 103 is operating in a low power mode.

The IMU 111 may be configured to detect when the electronic device 100 has been stationary but then begins to move, for example when left on a surface but then attached to the garment 200. The IMU 111 may be configured to detect that the wearer 600 of the garment 200, with the electronic device attached, is resting, or is moving, for example during exercise. The IMU 111 may be configured to establish the level of activity, for example, whether the wearer 600 is walking or running.

The IMU 111 communicates with the controller 103 over a serial protocol such as the Serial Peripheral Interface (SPI), Inter-Integrated Circuit (I2C), Controller Area Network (CAN), and Recommended Standard 232 (RS-232). Other serial protocols are within the scope of the present disclosure. The IMU 111 is also able to send interrupt signals to the controller 103 when required so as to transition the controller 103 from a low power model to a normal power mode when a motion event is detected, for example, or vice versa. The interrupt signals may be transmitted via one or more dedicated interrupt pins.

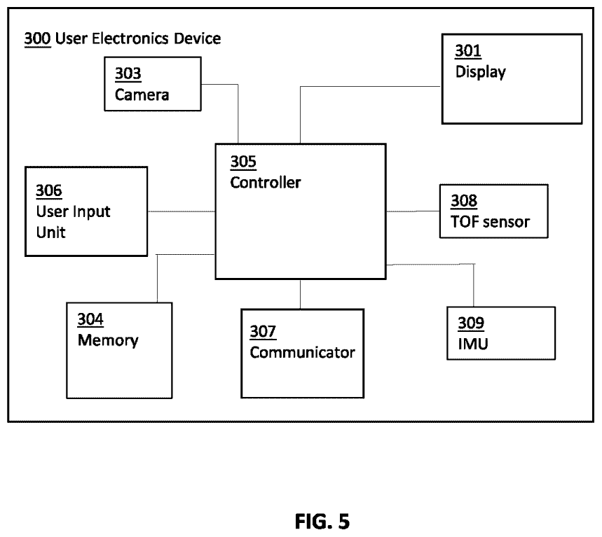

The user electronic device 300 in the example of FIG. 5 is in the form of a mobile phone or tablet and comprises a controller 305, a memory 304, a wireless communicator 307, a display 301, a user input unit 306, a capturing device in the form of a camera 303 and an inertial measurement unit (IMU) 309. The controller 305 provides overall control to the user electronic device 300.

The user input unit 306 receives inputs from the user such as a user credential.

The memory 304 stores information for the user electronic device 300.

The display 301 is arranged to display a user interface 302 for applications operable on the user electronic device 300.

The IMU 309 provides motion and/or orientation detection and may comprise an accelerometer and optionally one or both of a gyroscope and a magnetometer.

The user electronic device 300 may also include a biometric sensor. The biometric sensor may be used to identify a user or users of device based on unique physiological features. The biometric sensor may be: a fingerprint sensor used to capture an image of a user's fingerprint; an iris scanner or a retina scanner configured to capture an image of a user's iris or retina; an ECG module used to measure the user's ECG; or the camera of the user electronic arranged to capture the face of the user. The biometric sensor may be an internal module of the user electronic device. The biometric module may be an external (stand-alone) device which may be coupled to the user electronic device by a wired or wireless link.

The controller 305 is configured to launch an application which is configured to display insights derived from the biosignal data processed by the ADC front end 139 of the electronics module 100, input to electronics module controller 103, and then transmitted from the electronics module 100. The transmitted data is received by the wireless communicator 307 of the user electronic device 300 and input to the controller 305.

Insights include, but are not limited to, heart rate, respiration rate, core temperature but can also include identification data for the wearer 600 using the wearable assembly 500.

The display 301 is also configured to display an ECG signal trace part of the user interface 302. To display a signal trace may require raw ECG data from the electronic module 100.

The display 301 may be a presence-sensitive display and therefore may comprise the user input unit 306. The presence-sensitive display may include a display component and a presence-sensitive input component. The presence sensitive display may be a touch-screen display arranged as part of the user interface 302.

User electronic devices 300 in accordance with the present invention are not limited to mobile phones or tablets and may take the form of any electronic device which may be used by a user to perform the methods according to aspects of the present invention. The user electronic device 300 may be a electronics module such as a smartphone, tablet personal computer (PC), mobile phone, smart phone, video telephone, laptop PC, netbook computer, personal digital assistant (PDA), mobile medical device, camera or wearable device. The user electronic device 300 may include a head-mounted device such as an Augmented Reality, Virtual Reality or Mixed Reality head-mounted device. The user electronic device 300 may be desktop PC, workstations, television apparatus or a projector, e.g. arranged to project a display onto a surface.

In use, the electronics module 100 is configured to receive raw biosignal data from the sensors 209, 211 and which are coupled to the controller 103 via the interface 101 and the ADC front end 139 for further processing and transmission to the user electronic device 300 as described above. The data transmitted to the user electronics device 300 includes raw or processed biosignal data such as ECG data, heart rate, respiration data, core temperature, IMU data and other insights as determined, and as required.

The controller 305 of the user electronics device 300 is also operable to launch an application which is configured to receive, process and display data, such as raw or processed biosignal data, from the electronics module 100. A user, such as the wearer 600, is able to configure the application, using user inputs, to receive, process and display the received data in accordance with these user inputs.

The user electronic device 300 is arranged to receive the transmitted data from the electronics module 100 via the communicator 307 and which are coupled to the controller 305, and then to process and display the data in accordance with the user configuration.

The controller 305 of the user electronics device 300 is operable to display information to a user on the display 301 as part of the user interface 302. Information displayed can be an ECG trace as well using raw data points transmitted from the electronic module 100. Other insights and data can be displayed on the display 301 as part of the user interface 302 and as required. Examples might be a heart rate in beats per minute, core temperature data and respiration rate.

Typically, sensors 209, 211 such as those described above are provided as part of a chest-band that is held tightly against the skin surface. The sensors 209, 211 may also be provided with adhesive conductive gel pads to enable them to be coupled to the skin surface. These wearable articles are typically only worn in a clinical or performance setting and for a relatively short time period such as during a workout or assessment.

It is an objective of the present invention to provide sensors 209, 211 in other forms of wearable article that do not require the sensors 209, 211 to be held close to the heart and/or in tight contact with the skin surface. These wearable articles 200 may be, for example, loose fitting garments, wristbands or armbands that can be comfortably worn over an extended period of time including during sleep.

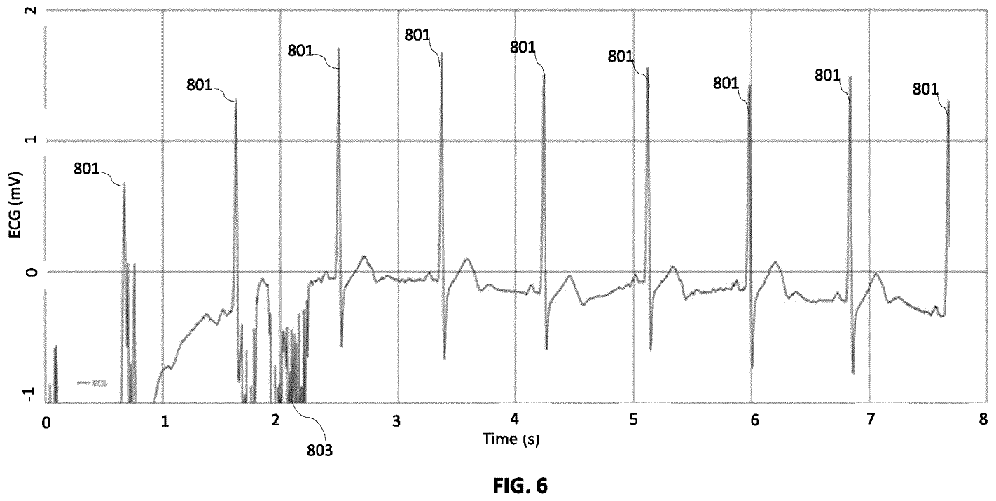

FIG. 6 shows an ECG trace obtained from sensors 209, 211 positioned on either side of the heart of a wearer and held tightly in place with the skin surface. The trace in FIG. 6 has clearly defined R-peaks 801 which can be used to determine measures such as the heart rate and heart rate variability of the wearer. Noise artefacts 803 are present, but the high amplitude of the R-peaks 801 means that they do not affect the peak detection process.

FIG. 7 shows an ECG trace obtained from sensors 209, 211 incorporated in a waist-band held around the waist of a wearer. Compared to the trace shown in FIG. 6, it will be appreciated that the R-peaks 805 are not as clearly defined relative to the other components of the signal. In addition, the signal suffers from a greater amount of noise due to baseline wander. Noise artefacts 807 can have an amplitude similar to the amplitude of the R-peaks which can make it difficult to accurately detect the true R-peaks 805.

While the ECG trace in FIG. 7 is of lower quality than the trace of FIG. 6 effective peak detection can still be performed as the peaks are still clearly identifiable in the signal.

The traces in FIGS. 6 and 7 were obtained when the wearer was sitting down. As such, there was limited motion of the wearer and limited motion artefacts in the resultant ECG signals. If the wearer in a high motion state such as if the wearer were running or performing other forms of exercise, motion artefacts would be introduced into the signals.

The presence of motion artefacts has a limited effect on signals obtained from the sensor configuration of FIG. 6 as the peaks have a sufficiently high amplitude that they can be easily identified and distinguished from peaks due to noise. Moreover, peak detection algorithms can include forms of motion compensation and filtering to remove or mitigate the effect of motion.

However, due to the low amplitude signal peaks and baseline wander for the sensor configuration of FIG. 7, the presence of motion artefacts can dramatically diminish the effectives of the peak detection algorithm in identifying peaks in the ECG signal. The true R-peaks in the signal can be surrounded by other peaks caused by motion to the extent that they cannot be isolated and identified by the algorithm.

A similar affect is observed when the sensors 209, 211 are not held tightly in place with the skin surface as increased motion can cause the sensors 209, 211 to move relative to the skin surface which decreases the quality of the sensed signal rendering peak detection difficult. This means that effective sensing can only be performed in situations where the sensors 209, 211 are displaced from the heart and/or not held tightly against the skin when the user is in a low motion state such as when they are sitting, standing or engaging in light activity such as walking.

While it would be possible to continually record and store signals from the sensors 209, 211 and discard poor quality data in post-processing, this would increase the power consumption and memory consumption of the electronics module 100 which would limit the operational life of the electronics module 100 before it needs recharging or to transfer data to an external device. This would limit the effectiveness of the electronics module 100 in performing monitoring of the wearer 600 of an extended time period such as during the day and night.

The present disclosure overcomes the above problems using contextual data to determine whether the wearer 600 is in a first motion state or a second motion state representative of a higher degree of activity of the wearer 600 than the first motion state. When the wearer 600 is in the first motion state, a measurement is performed using the sensors 209, 211.

This means that measurements are performed and stored when the wearer 600 is determined to be in a low motion state. This indicates that the wearer 600 is in a resting position and that the likelihood of their being motion artefacts in the measured signal is low. The issues associated with motion artefacts for sensors 209, 211 that are displaced from the heart or not held tightly in contact with the skin are therefore avoided. Moreover, the present disclosure does not require continuously performing and storing measurements regardless of wearer motion which avoids the problems of increased power and memory consumption described above.

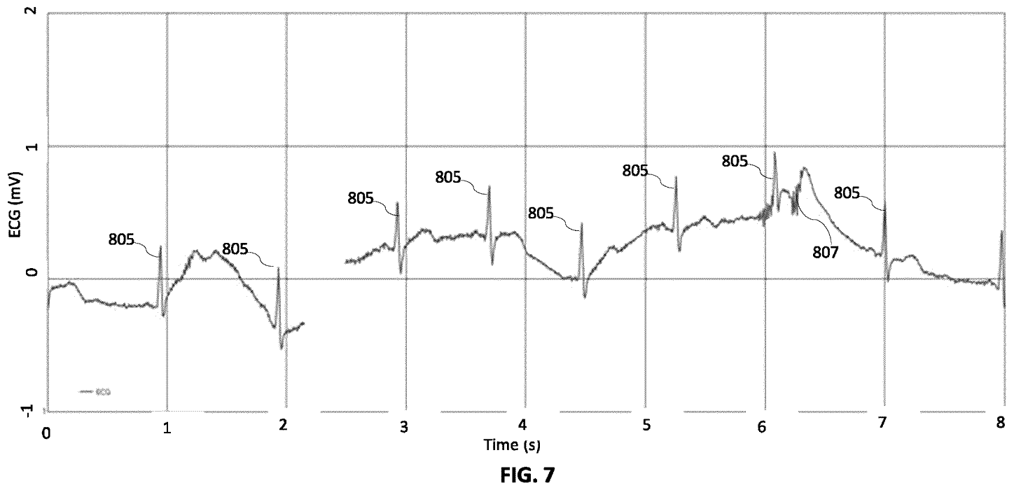

Referring to FIG. 8, there is shown a flow diagram for an example method according to aspects of the present disclosure. The method is performed by the controller 103 of the electronics module 100.

In step S101 the controller 103 enters a first measurement mode.

In step S102, the controller 103 accesses contextual data. The contextual data is indicative of whether the wearer 600 of the wearable article 200 is in a first motion state or a second motion state. The second motion state is representative of a higher degree of activity of the wearer than the first motion state.

The first motion state is a state of generally low motion by the wearer 600 where the sensors 209, 211 are able to measure accurate and reliable signals from the body surface. The first motion state may occur when the wearer 600 is lying down, sitting, standing, walking or engaging in forms of low impact exercise.

The second motion state is a state of generally high motion by the wearer 600 where the sensors 209, 211 are generally not able to measure accurate and reliable signals from the body surface. The second motion state may occur when the wearer 600 is jogging, running, jumping, cycling or engaging in other forms of high impact exercise.

It will be appreciated that the boundary between the first motion state and the second motion state is not rigidly fixed and can vary depending on the type of sensor, the type of wearable article, the fit of the wearable article and how tightly it holds the sensor against the skin surface, and the position of the sensor on the wearable article.

In some examples, the sensors 209, 211 are held loosely with respect to the body surface. This may occur when the sensors 209, 211 are incorporated into a loose-fitting shirt. In these examples, the first motion state may only include states with very little motion of the wearer such as when the wearer is lying down, sitting or standing. This enables effective sleep monitoring or monitoring when the wearer is working at a desk or relaxing. Effective measurements may not be obtainable when the wearer 600 is walking, running or engaging in other forms of exercise.

In other examples, the sensors 209, 211 are more tightly held such as when incorporated in a waistband of a pair of trousers, leggings or underwear. The waistband helps ensure more effective contact between the sensors and the skin surface for a greater range of activities such as walking, jogging, and yoga, but may cease to be effective in more intensive activities such as running.

The contextual data may be any form of data that indicates the motion state of the wearer 600.

In some examples, the contextual data comprises motion data for the wearer 600. The motion data may be obtained from the motion sensor 111 of the electronics module 100. The motion sensor 111 may thus operate as a contextual data unit.

The controller 103 may receive motion signals from the motion sensor 111 such as accelerometer signals or may receive an inferred output from a machine-learning core of the motion sensor 111 as described above.

In some examples, the contextual data comprises location data for the wearer 600. The location data may be obtained from the location device 161 of the electronics module 100. The location device 161 of the electronics module 100 may thus operate as a contextual data unit. The location data may also be received from a location device of another apparatus in communication with the electronics module 100 such as the user phone 300.

The controller 103 may use cues from the location data to determine the likely motion state of the wearer 600. For example, if the location data indicates that the wearer 600 is at home, close to a desk or workstation (e.g. at home or in an office), or close to a bedroom, then the controller 103 may determine that the wearer 600 is in the first motion state. Likewise, if the location data indicates that the wearer 600 is at a gym, pitch, or swimming pool or other location associated with high impact exercise, then the controller 103 may determine that the wearer 600 is in the second motion state.

In some examples, the contextual data comprises an input received from a user. The user may be the wearer 600 of the wearable article 200 or another person. The information may indicate the motion state of the wearer 600 such as whether the wearer 600 is in the first motion state or the second motion state.

In an example implementation, the user may access an application running on the user electronic device 300 and input, via the application, that the wearer 600 is in the first motion state. The user electronic device 300 sends this information to the electronics module 100 to inform the controller 103 that the wearer is in the first motion state. Equally, the user may input that the wearer 600 is in the second motion state.

In step S103, the controller 103 determine from the contextual data whether the wearer 600 of the wearable article 200 is in the first motion state.

If the wearer 600 is in the first motion state, the method proceeds to step S104. Otherwise, the method returns to step S102. If after a certain period of time, the controller 103 is still unable to confirm that the wearer 600 is in the first motion state, then the controller 103 may exit the first measurement mode.

In step S104, the controller performs a measurement using the sensors 209, 211. In ECG sensing, this could mean performing a differential voltage measurement using a pair of sensors 209, 211. The differential voltage measurement could be performed using the ADC front end 139 described above in relation to FIG. 4. This could mean performing a bioimpedance measurement performed by injecting a current into body of the wearer using the sensors 209, 211 and measuring the resultant voltage. The bioimpedance measurement could be performed by the ADC front end 139. For other examples of sensors 209, 211 the measurements can be performed in different ways.

The controller 103 may enter the first measurement mode according to a predetermined schedule.

For example, once every twenty minutes, the controller 103 may enter the first measurement mode. While in the first measurement mode, the controller 103 monitors the contextual data until the controller 103 determines that the wearer 600 is in the first measurement mode. The controller 103 then performs one or more measurements using the sensors 209, 211. Other schedules are possible as will be appreciated by the skilled person. The controller 103 may enter the first measurement mode once every thirty minutes, hour, two hours, six hours for example.

In some examples, the controller 103 uses one or more of the measurements performed in the first measurement mode to configure an algorithm, which receives, as input, one or more measurements from the sensors 209, 211. As the controller 103 knows that the measurements are performed while the wearer is in a low motion state, the controller 103 knows that these measurements are likely to be accurate and reliable and so can use them as baseline or reference values to update the algorithm.

In one example, the algorithm comprises a peak detection algorithm arranged to detect peaks in a measurement signal received from the sensors 209, 211. The controller 103 is operable to configure a filtering or threshold parameter for the peak detection algorithm using the measurement(s) performed in the first measurement mode. Such peak detection algorithms are known in the art so their details are omitted. The present disclosure is not limited to adjusting peak detection algorithms and other algorithms are within the scope of the present disclosure.

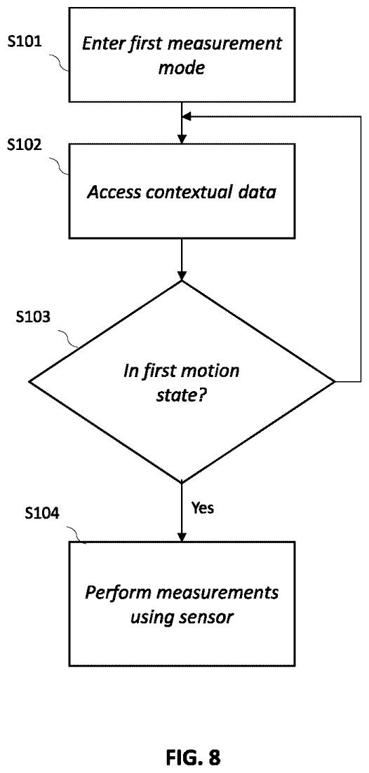

Referring to FIG. 9, there is shown a flow diagram for an example method according to aspects of the present disclosure. The method is performed by the controller 103 of the electronics module 100.

In step S201 the controller 103 enters a first measurement mode.

In step S202, the controller 103 accesses contextual data. The contextual data is indicative of whether the wearer 600 of the wearable article 200 is in a first motion state or a second motion state. The second motion state is representative of a higher degree of activity of the wearer than the first motion state.

In step S203, the controller 103 determine from the contextual data whether the wearer 600 of the wearable article 200 is in the first motion state.

If the wearer 600 is in the first motion state, the method proceeds to step S204. Otherwise, the method returns to step S202. If after a certain period of time, the controller 103 is still unable to confirm that the wearer 600 is in the first motion state, then the controller 103 may exit the first measurement mode.

In step S204, the controller performs a plurality of measurement using the sensors 209, 211 over a first predetermined time period. The plurality of measurements provide a snap-shot of the health state of the wearer. The predetermined time period may be any time period as selected by the skilled person. The predetermined time period may be 10 seconds or more, 20 seconds or more, 30 seconds or more, 1 minute or more, 2 minutes or more, 5 minutes or more or 10 minutes or more for example.

In step S205, the controller 103 determines whether the wearer has remained in the first motion state over the first predetermined time period. The controller 103 performs this determination using contextual data as described above. If the wearer is determined to not have remained in the first motion state during the first time period, the method returns to step S202. If the wearer is determined to have remained in the first motion state during the first time period, the method proceeds to step S206.

In step S206, the controller 103 stores the measurements performed over the first time period in a memory. The memory may be a memory of the controller or a separate memory of the wearable article.

Advantageously, according to this method, the controller performs a plurality of measurements over the first predetermined time period in response to determining that the wearer is in the low motion state. This enables a snapshot reading of the wearer to be performed. If the wearer remains in the low motion state throughout the measurement period, the snapshot measurement is saved. This avoids excess power consumption associated with performing measurements continually regardless of the motion state of the wearer and also avoids excess memory consumption associated with storing measurements regardless of the motion state of the wearer.

In one example of this method, the controller 103 may enter the first measurement mode according to a predetermined schedule such as once every 30 minutes. While in the first measurement mode, the controller will perform the plurality of measurements over a time period of 30 seconds. If the wearer remained in the first motion state over the 30 second time period, then the snapshot measurement is saved. The controller may then transition to a low power state until it is next scheduled to enter the first measurement mode.

During the performance of this method, wireless communication capabilities are not necessarily required to be activated for the wearable article. The controller 103 is not required to control a wireless communicator to transmit the measurements to an external device and instead the measurements can just be stored locally in a memory. This reduces power consumption. By performing this method, monitoring of a wearer is able to be performed over extended time periods without requiring a power source of the wearable article to be replaced or recharged. In some examples, performing measurements for 30 seconds once every 30 minutes will allow the controller to operate in the region of 15 days without requiring a power source to be replaced or recharged.

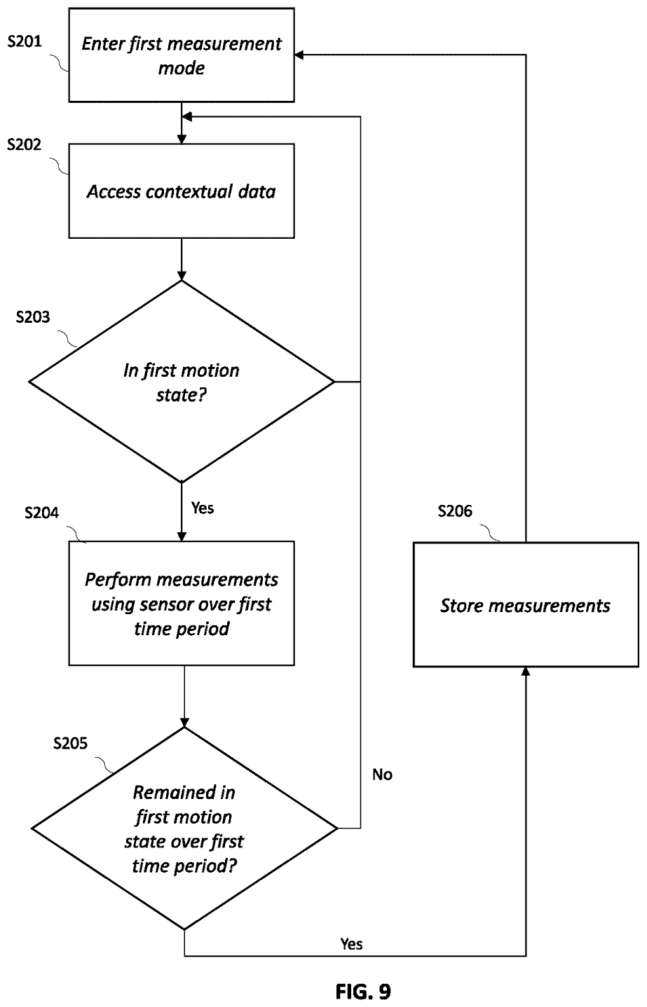

Referring to FIG. 10, there is shown a flow diagram for a example method according to aspects of the present disclosure. The method is performed by the controller 103 of the electronics module 100.

In step S301 the controller 103 enters a first measurement mode.

In step S302, the controller 103 access contextual data. The contextual data is indicative of whether the wearer 600 of the wearable article 200 is in a first motion state or a second motion state. The second motion state is representative of a higher degree of activity of the wearer than the first motion state.

In step S303, the controller 103 determine from the contextual data whether the wearer 600 of the wearable article 200 has been in the first motion state for more than a first predetermined time period.

The predetermined time period may be any time period that indicates that the wearer 600 is not just temporarily in the low motion state. The predetermined time period may be 10 seconds or more, 20 seconds or more, 30 seconds or more, 1 minute or more, 2 minutes or more, 5 minutes or more or 10 minutes or more for example.

If the wearer 600 is in the first motion state for more than the first predetermined time period, the method proceeds to step S304. Otherwise, the method returns to step S302. If after a certain period of time, the controller 103 is still unable to confirm that the wearer 600 has been in the first motion state for more than the first predetermined time period, then the controller 103 may exit the first measurement mode.

In step S304, the controller performs a measurement using the sensors 209, 211.

In step S305, the controller 103 determines whether an exit condition has been reached. If an exit condition has been reached, the controller 103 returns to step S301. Otherwise, the controller 103 repeats step S304 and performs one or more additional measurements using the sensors 209, 211. In this way, the controller 103 repeats performing measurement using the sensors 209, 211 until the exit condition is reached.

The exit condition may be a time-based exit condition. That is, the controller 103 may repeatedly perform measurements until a second predetermined time period has elapsed. The second predetermined time period may be any time period as desired by the skilled person. For example, the second predetermined time period may be 1 minute, 2 minutes, 5 minutes, 10 minutes 20 minutes, 30 minutes or 1 hour.

The exit condition may be an iteration-based exit condition. That is, the controller 103 may repeatedly perform measurements until a predetermined number of measurements have been performed. Any predetermined number of measurements may be selected as desired by the skilled person based on, for example, a desired measurement rate of the sensors 209, 211.

The exit condition may be reached if the controller 103 determines that the wearer 600 has moved from the first motion state to the second motion state.

The exit condition may be reached if the controller 103 determines that the measurement signals received from the sensors 209, 211 do not satisfy one or more quality criterion. Example quality criterions include the signal amplitude and the signal-to-noise ratio.

The exit condition may be reached if the controller 103 determines that the sensors 209, 211 are no longer in contact with the body surface. This may be caused by the wearable article 100 being removed from the wearer 600, for example.

Any or a combination of the above exit conditions may be used.

The controller 103 may enter the first measurement mode according to a predetermined schedule.

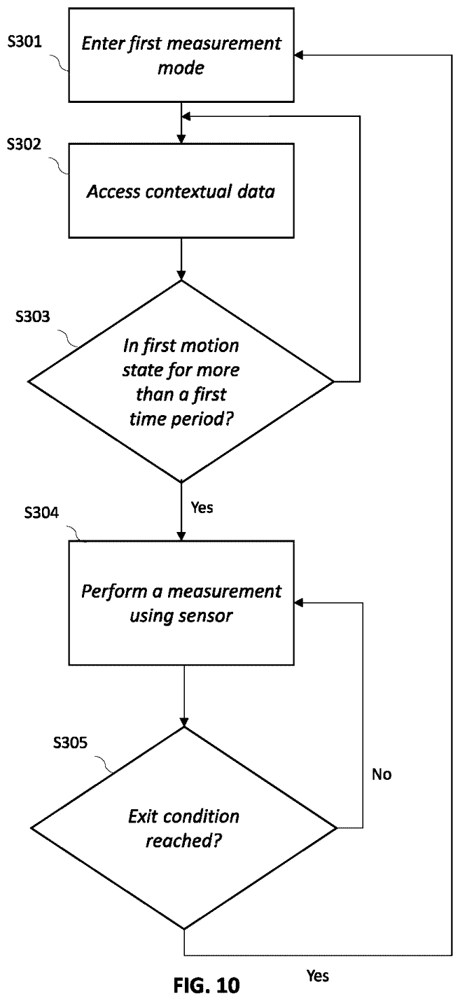

Referring to FIG. 11, there is shown another example method according to aspects of the present disclosure. The method is performed by the controller 103 of the electronics module 100.

In step S401, the controller 103 enters a first measurement mode.

In step S402, the controller 103 determines whether the wearable article 200 is being worn.

The controller 103 may determine that the wearable article 200 is being worn in response to receiving a signal from the sensors 209, 211 or other sensors of the electronics module 100 or the wearable article 200. Particular examples include sensor data obtained from bioelectrical, bioimpedance, optical, temperature or humidity sensors. Generally, bioelectrical or bioimpedance sensors such as electrocardiogram (ECG) and electromyography (EMG) sensors will generate signals with characteristics properties when the sensors are in contact with the body surface of the wearer 600. For example, when ECG electrodes are in contact with a skin surface of the wearer 600 they generate a distinctive voltage profile that can be used to determine that the electronics module 100/garment 200 is being worn. Similarly, photoplethysmography (PPG) sensors will generate distinctive signals when recording signals from a user. Temperature sensors such as skin surface temperate or core body temperature sensors and humidity sensors will also output temperature/humidity values that are indicative of whether the electronics module 100/garment 200 is being worn. Generally, any form of sensor that can generate signals indicative of whether the electronics module 100/garment 200 is being worn can be used in accordance with the present disclosure. Detecting whether the electronics module 100/garment 200 is being worn is generally the same as performing liveness detection.

In one example, the analog front end 139 of the electronics module 100 may detect a potential difference change caused by the sensors 209, 211 contacting the body surface of the wearer 600. This may be referred to as a body-detect signal or a leads-on signal. The analog front end 139 may generate an interrupt signal in response to detecting the input event which is received by the controller 103 and used to determine that the wearable article 200 is being worn.

In other examples, the controller 103 may determine that the wearable article 200 is worn in response to receiving an input by the user. This may be caused by the user tapping against the electronics module 100 such as with their phone 300. The tap event may be detected by the motion sensor 111 or the NFC antenna 107 if the phone 300 has an antenna which is energized to induce a current in the NFC antenna 107. The input may also be received from the user electronic device 300 as a result of the user inputting information into an application running on the user electronic device 300. For example, the user electronic device 300 may press a button on an interface of the user electronic device 300 to indicate that the wearable article 200 is being worn.

If the wearable article 200 is determined to be worn, the controller 103 proceeds to step S403. Otherwise, the controller 103 repeats step S402. If after a certain period of time, the controller 103 is still unable to confirm that the wearable article 200 is being worn, then the controller 103 may exit the first measurement mode.

In step S403, the controller 103 access contextual data. The contextual data is indicative of whether a wearer 600 of the wearable article 200 is in a first motion state or a second motion state. The second motion state is representative of a higher degree of activity of the wearer 600 than the first motion state.

In step S404, the controller 103 determines from the contextual data whether the wearer 600 of the wearable article 200 is in the first motion state

If the wearer 600 is in the first motion state, the method proceeds to step S405. Otherwise, the method returns to step S403. If after a certain period of time, the controller 103 is still unable to confirm that the wearer 600 has been in the first motion state for more than the first predetermined time period, then the controller 103 may exit the first measurement mode.

In step S405, the controller performs a measurement using the sensors 209, 211.

When entering the first measurement mode, the electronics module 100 may be operating in a first power mode. In response to detecting that the wearable article is being worn in step S402, the controller 103 may transition the electronics module 100 to a second power mode that consumes more power than the first power mode. In this way, power consumption is reduced until the electronics module 100 detects that the wearable article 200 is being worn.

In the first power mode, the controller 103 may be unable to perform measurements using the sensors 209, 211. Instead, the controller 103 (or the electronics module 100 more generally) may only be able to perform simple leads-on/body detection to look for a potential difference change caused by the sensors 209, 211 being placed in contact with the body surface.

In general, in the first power mode, most components of the electronics module 100 are disabled or otherwise operating in a low power state. Certain components such as motion sensor 111 may be enabled to perform tap detection such as for detecting whether the wearable article 200 is being worn as described above, but may not be operating at their full capacity and advanced functions such as provided by the machine-learning core may be disabled. The communicator 159 may be disabled, may be operating only in a receive mode, or may only be selectively enabled to transmit/receive information (e.g. according to a schedule). The communicator 159 may be disabled from transmitting measurement data to the user electronic device 300.

In the second power mode, the components of the electronics module 100 are generally enabled and operating in their normal mode.

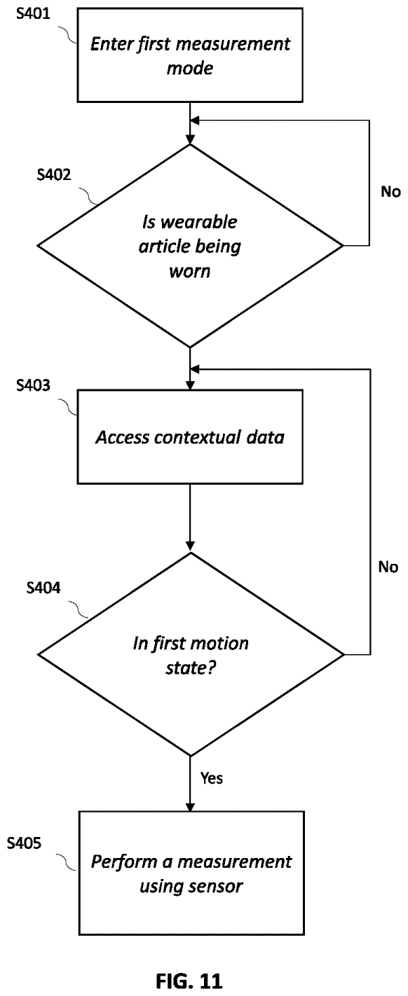

Referring to FIG. 12, there is shown a flow diagram for another example method according to aspects of the present disclosure. The method is performed by the controller 103 of the electronics module 100.

In step S501, the controller 103 accesses contextual data. The contextual data may be any of the contextual data as described above.

In step S502, the controller 103 determines, from the contextual data, whether to use the first measurement mode or a second measurement mode. If the controller 103 determines to enter the first measurement mode, the method proceeds to step S503. If the controller 103 determines to enter the second measurement mode, the method proceeds to step S504.

In step S503, the controller 103 enters the first measurement mode. The first measurement mode may the modes described above in relation to FIGS. 8 to 11.

In step S504, the controller 103 enters the second measurement mode. In the second measurement mode, the controller 103 repeatedly performs measurements using the sensors 209, 211 of the wearable article 200 regardless of the motion state of the wearer 600. This may mean that the controller 103 performs measurements using the sensors 209, 211 at a first measurement rate independent of the motion state of the wearer 600. This enables the controller 103 to continuously obtain measurements from the sensors 209, 211.

In some examples, the electronics module 100 is able to communicatively couple with different wearable articles comprising sensors. Some of the wearable articles may be loose fitting and/or have sensors positioned away from the chest region of the wearer. These wearable articles may be intended to be comfortable daywear or nightwear. Other wearable articles may be more tight fitting and/or have sensors positioned close to the chest region of the wearer. These wearer articles may be athletic clothing intended to be worn during exercise.

The controller 103 may determine, from contextual data, the type of wearable article it is coupled to. If the controller 103 determines that it is coupled to an item of daywear or nightwear then it enters the first measurement mode. By contrast, if the controller 103 determines that it is coupled to an item of athletic clothing then it enters the second measurement mode. Advantageously, this means that the controller 103 can switch between the measurement modes it operates in based on the type of wearable article it is coupled to.

The contextual data used may comprise motion data as described above. The motion data may comprise the orientation or vertical position of the electronics module 100 when coupled to the wearable article. The orientation/position of the electronics module 100 may be unique to a certain type of wearable article. For example, if the controller 103 determines that it is positioned on the chest of the wearer, then it may determine that it is coupled to an item of athletic clothing and enter the second measurement mode. By contrast, if the controller 103 determines that it is positioned on the waist of the wearer, then it may determine that it is coupled to an item of daywear/nightwear and enter the first measurement mode.

The contextual data may comprise location data as described above. The controller 103 may associate different locations with different types of wearable articles. If for example, the controller 103 determines that the wearer is located at home, a workplace, or a bedroom, it may determine that it is coupled to an item of daywear/nightwear and enter the first measurement mode. By contrast, if the controller 103 determines that it is located at a gym, swimming pool, track or stadium, it may determine that it is coupled to an item of athletic clothing and enter the second measurement mode.

The contextual data may comprise an input received from the user indicating the desired measurement mode. The input may be received from the user electronic device 300 as described above.

An example of a user case scenario is provided below in relation to the examples of FIGS. 8 to 12:

The wearer 600 wakes up and dons an item of daywear 200 incorporating sensors 209, 211 and inserts the electronics module 100 into a pocket 201 of the clothing 200. The controller 103 of the electronics module 100 determines that it is coupled to daywear and enters the first measurement mode.

Initial measurements performed in the first measurement mode such as when the wearer 600 is sitting or lying down are used as baseline measurements to configure one or more algorithms such as the peak detection algorithm described above.

The wearer 600 takes the train to the gym. During the wearer's travelling to the gym, the controller 103 enters the first measurement mode according to a schedule and performs measurements if the motion conditions are satisfied. This provides insights relating to the wearer's health status.

The wearer 600 heads to the gym and changes to an athletic garment 200 incorporating sensors 209, 211. The wearer 600 removes the electronics module 100 from the daywear and inserts the electronics module 100 into a pocket 201 of the athletic garment 200. The controller 103 determines that it is coupled to athletic clothing 200 and enters the second measurement mode. During the second measurement mode, the controller 103 repeatedly performs measurements using the sensors 209, 211.

The wearer 600 changes into their work clothing 200 which also incorporates sensors 209, 211. The wearer 600 removes the electronics module 100 from the athletic clothing 200 and inserts the electronics module 100 into a pocket 201 of the work clothing 200. The controller 103 determines that it is coupled to work clothing 200 and enters the first measurement mode.

The work clothing 200 in this example is a loose-fitting shirt 200. The wearer walks 600 to work and the controller 103 determines that the motion state is high and thus in the second motion state. The controller 103 therefore does not perform measurements as the wearer 600 walks to work.

At the office, the wearer 600 sits at their workstation. The controller 103 determines that the wearer 600 is in the first motion state and performs measurements using the sensors 209, 211. Measuring the wearer 600 while at the desk can be important for detecting stress levels of the wearer 600.

The wearer 600 returns home and changes into sleepwear 200 which also incorporates sensors 209, 211. The wearer 600 removes the electronics module 100 from the work clothing 200 and inserts the electronics module 100 into a pocket 201 of the sleepwear 200. The controller 103 determines that it is coupled to sleepwear 200 and enters the first measurement mode.

As the wearer 600 is sleeping, the controller 103 determines that the wearer 600 is in the first motion state and performs measurements using the sensors 209, 211. This enables the controller 103 to perform sleep tracking operations.

Advantageously, the controller 103 is able to adapt its operation based on the type of wearable article 200 it is coupled to and the type of activity performed by the wearer 600. This enables the controller 103 to control the performance of measurements using the sensors 209, 211 so that they are most suited for the particular situation. This adaptive control of the measurements performed by the sensors 209, 211 helps avoids recording unusable data and avoids unnecessary power and memory consumption.

The present disclosure is not limited to electronics modules 100 that communicatively couple with sensors 209, 211 incorporated into the wearable article 200. In some examples, the electronics module 100 may use an internal sensor to perform the measurements. The wearable article 200 may not comprise any sensing units.

The internal sensor may be an optical sensor. The optical sensor may measure light in one or more of the infrared, visible, and ultraviolet spectrums. The optical sensor may be a pulse oximeter. The optical sensor may be arranged to measure the oxygen saturation of the wearer. Oxygen saturation is the fraction of oxygen-saturated haemoglobin relative to total haemoglobin (unsaturated+saturated) in the blood. The optical sensor may be arranged to measure the capillary perfusion of the wearer. A pulse oximeter may be useable to measure the capillary perfusion using a double-wavelength method. The capillary perfusion can be derived from a variation in the detected signal strength. The optical sensor may be arranged to measure the temperature of the wearer.

The sensor is not required to comprise an optical sensor in all examples. The sensor is generally arranged to monitor a property of the environment external to the electronics module. The property may be a property of the user wearing the garment. The sensor 604 may comprise one or more of an altitude sensor, pressure sensor, temperature sensor, optical sensor, humidity sensor, presence sensor, and air quality sensor. The presence sensor may for detecting a touch input from a user. The presence sensor may comprise one or more of a capacitive sensor, inductive sensor, and ultrasonic sensor.

The sensor may comprise an infrared temperature sensor arranged to measure the skin surface temperature of a user wearing the wearable article. The temperature sensor may be an ambient temperature sensor

The wearable article 200 may be constructed such that, when coupled to the wearable article, the internal sensor has line of sight with a skin surface of the wearer. The wearable article 200 may comprise an opening or window to enable the internal sensor to have line of sight with the skin surface. The window may be constructed from a transparent, translucent, or light diffracting material. The use of a light diffracting material may provide a light pipe effect.

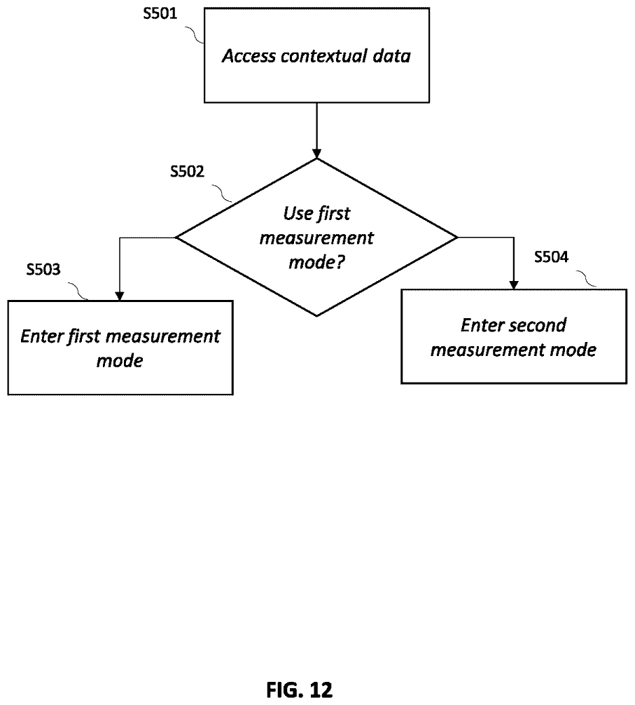

Referring to FIG. 13, there is shown another example wearable assembly 500 in accordance with aspects of the present disclosure. The wearable assembly 500 comprises a wearable article 200 in the form of a band 200 such as a chest band, waist band, arm band, wrist band or bra underband. The band 200 may be a stand-alone unit or may be formed as part of a garment such as a pair of trousers, leggings, shorts, underwear or a bra.

The band 200 comprises a layer of fabric material that is arranged to surround the circumference of the wearer 600. In this example, the band 200 is adjustable in length through the use of sliders 217 or other fasteners. These are not required in all examples. The layer of fabric material may be formed of a stretch material (elastomeric material) such that it can be stretched over and tighten against the wearer 600. The band 200 may be formed from a continuous loop of material but this is not required in all examples.

The band 200 comprises a sensing unit 400 comprising a pair of sensors 209, 211 in the form of electrodes 209, 211 which are connected to connection regions 213, 215 by conductive pathways 203, 207. The sensors 209, 211 are provided on the insider surface of the layer of fabric material such that they contact the skin surface when worn. The sensors 209, 211, connection regions 213, 215, and conductive pathways 203, 207 in this example are formed from conductive yarn which is knitted integrally with the layer of fabric material. Other forms of conductive material may also be used.

The band 200 is arranged such that the connection regions 213, 215 are positioned proximate to one another. The electrodes 209, 211 are spaced apart from one another. The connection regions 213, 215 are provided on an outer facing surface of the band 200. The electrodes 209, 211 are provided on an inner facing surface of the band 200.

A pocket layer 201 is attached to the band by side seams 219, 221. The pocket layer 201 forms a pocket space between the pocket layer 201 and the band 200 sized to removably receive electronics module 100. The upper margin of the pocket layer 201 is unattached to the band to provide an opening for the pocket space. The pocket layer 201 may be integrally formed with the band 200. The pocket layer 201 is not required and other forms of electronics module holder as described above may be used.

The band 200 further comprises a waterproof layer 223 attached to the band 200 by an adhesive layer 225. The waterproof layer 223 is a liquid impermeable layer. The waterproof layer 223 is provided in the pocket space between the band 200 and the pocket layer 201. The waterproof layer 223 prevents or otherwise restricts the ingress of water, such as due to sweat, into the pocket space from the body surface when the band 200 is worn. The waterproof layer 223 is formed from a waterproof film of material.

The waterproof layer 223 comprises recesses that are aligned with the connection terminals 213, 215 of the band 200. The recesses are openings that extend through the waterproof layer 223. Corresponding recesses are provided in the adhesive layer 225.

The pocket space is sized to removably receive an electronics module 100. When the electronics module 100 is positioned in the pocket space, the recesses enable the interface elements of the electronics module 100 to form a communicative connection with the connection terminals 213, 215. The communicative connection may be a conductive connection between the interface elements of the electronics module 100 and the connection terminals 213, 215. Other forms of communicative coupling such as wireless (e.g. inductive) are within the scope of the present disclosure.

The electronics module 100 may control the perform of measurements using the sensors 209, 211 according to the motion state of the wearer as described above in relation to FIGS. 8 to 10. In particular, the controller 103 of the electronics module 100 obtains contextual data indicative of whether the wearer is in a first, low, motion state or a second, high, motion state and determines the motion state of the wearer from this contextual data. When the wearer is in the first, low, motion state, the controller 103 performs one or more measurements using the sensors 209, 211. When in the second, high, motion state, the controller does not perform measurements using the sensors 209, 211.

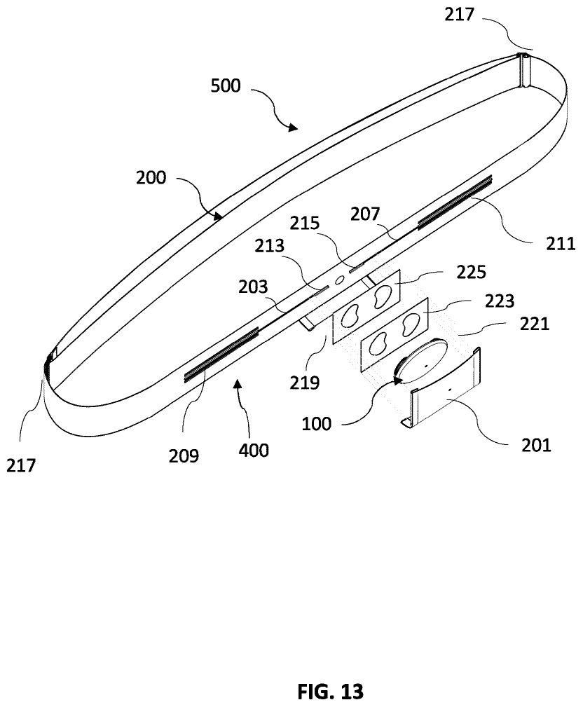

Referring to FIGS. 14 and 15, there are shown front and back views of an example wearable article 200 in accordance with aspects of the present disclosure.

The wearable article 200 is in the form of a shirt, and in-particular a t-shirt. The shirt 200 comprises a sensing unit 400 comprising a pair of sensors 209, 211 in the form of electrodes arranged to measure a signal from a body surface of the wearer.

The shirt 200 is a loose-fitting garment rather than a compression garment or other form of tight-fitting garment. Because of this, the sensors 209, 211 are able to move relative to the body surface particularly as a result of motion by the wearer of the shirt 200. This motion causes the sensors 209, 211 to move relative to the body surface of the wearer when the wearer is in high motion states. However, when the wearer is in a stationary or other low motion state, the sensors 209, 211 are generally fixed relative to the body surface and able to measure an accurate and reliable signal.

The electronics module 100, which is communicatively coupled to the wearable article 200, controls the measurement of signals using the sensors 209, 211 according to the motion states of the wearer as describe above in relation to FIGS. 8 to 12. In particular, the controller 103 of the electronics module 100 obtains contextual data indicative of whether the wearer is in a first, low, motion state or a second, high, motion state and determines the motion state of the wearer from this contextual data. When the wearer is in the first, low, motion state, the controller 103 performs one or more measurements using the sensors 209, 211. When in the second, high, motion state, the controller does not perform measurements using the sensors 209, 211.

The shirt 200 in FIGS. 14 and 15 comprises a front area 227 arranged to cover at least part of the front of the wearer, a back area 229 arranged to cover at least part of the back of the wearer, and a pair of shoulder regions 231, 233 connecting the front area 227 to the back area 229. A collar region 235 is provided between the pair of shoulder regions 231, 233 and defines a neck opening. The collar region 235 may be a simple neck opening as shown in FIGS. 14 and 15 or may comprise a collar stand and collar if the shirt is a polo-shirt, causal shirt or dress shirt. A pair of sleeves 237, 239 extend from the shoulder regions 231, 239 to cover at least part of the arms of the wearer.

The sensors 209, 211 are provided in the shoulder regions 231, 233 in this example. Providing the sensors 209, 211 in the shoulder regions 231, 233 is advantageous as the weight of the shirt 200 helps pull the sensors 209, 211 down into contact with the shoulders of the wearer. This helps ensure contact between the sensors 209, 211 and the body surface at least when the wearer is in a low motion state. The sensors 209, 211 may also have a three-dimensional profile to help ensure contact with the body surface. Moreover, the shoulders of the wearer are less affected by motion than other body parts such as the arms of the wearer which means that the sensors 209, 211 are less affected by wearer motion than at other positions on the wearable article 200. This means that the sensors 209, 211 are less affected by wearer motion and can be used to perform motions over a greater range of wearer motions than other sensor positionings. The sensors 209, 211 may also have a three-dimensional profile to improve contact with the body surface. However, selectively performing measurements as described above such that measurements are not performed in high motion states of the wearer is still beneficial in avoiding power consumption and memory consumption issues associated with performing measurements when the sensors 209, 211 are not in an optimum position relative to the body surface.

A pocket 201 is provided on the back area of the shirt. The pocket 201 is arranged to removably retain the electronics module 100. Conductive pathways 203, 207 extend from the sensors 209, 211 to connection regions in the pocket 201. In this way, the electronics module 100, when positioned in the pocket 201, is communicatively coupled to the sensors 209, 211 via the conductive pathways. Other forms of communicative coupling such as wireless (e.g. inductive) are within the scope of the present disclosure.

Providing the pocket 201 on the back area 229, and particularly on the upper back area close to the neck of the wearer and between the collar bones is advantageous particularly when the wearable article 200 is worn in high impact/combat sports. In this position, the electronics module 100 is unlikely to affect the comfort of the wearer if they fall or experience another form of impact such as a tackle or a collision in a team sport activity.

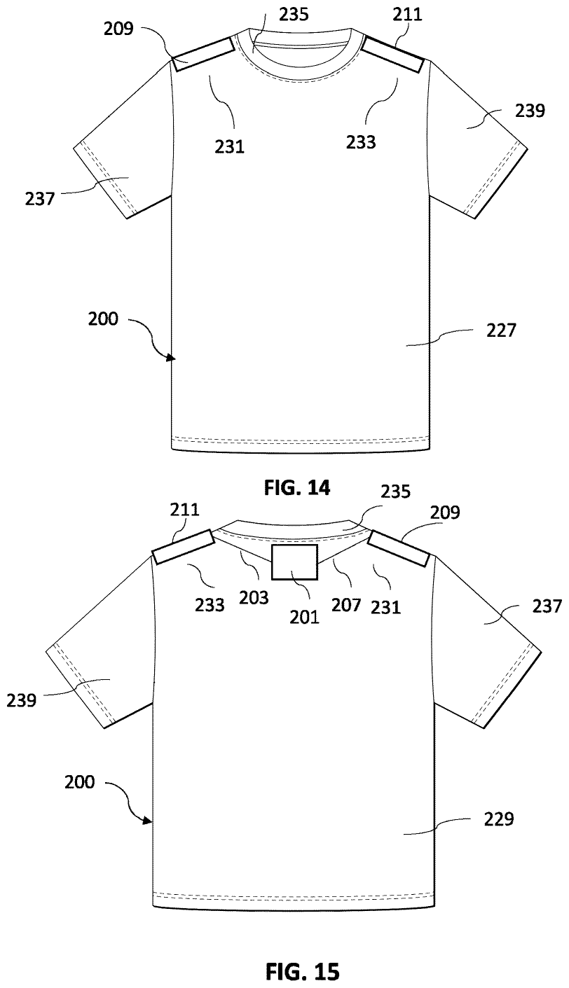

Similar affects can be achieved by positioning the pocket 201 on the collar region 235 as shown in FIG. 16. The sensors 209, 211 are not required in this example. The electronics module 100 may perform measurements using an internal sensor as described above and the collar region 235 may be constructed such that the internal sensor has line of sight with the skin surface of the wearer.

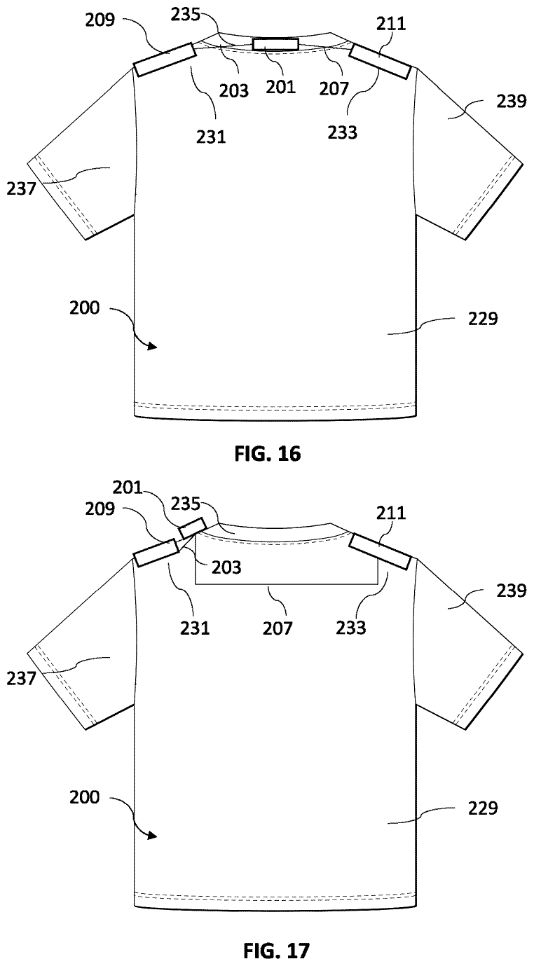

Similar affects can be achieved by positioning the pocket 201 on one of the shoulder regions 231, 233 as shown in FIG. 17. The sensors 209, 211 are not required in this example. The electronics module 100 may perform measurements using an internal sensor as described above and the shoulder region 231, 233 may be constructed such that the internal sensor has line of sight with the skin surface of the wearer.

The present disclosure is, however, not limited to pockets 201 positioned on the upper back area 229, collar region 235, or shoulder region 231, 233 of the wearable article 200 and may be positioned elsewhere on the back area 229, front area 227, or other part of the wearable article 200. The pocket 201 is not required and other forms of electronics module holder as described above may be used.

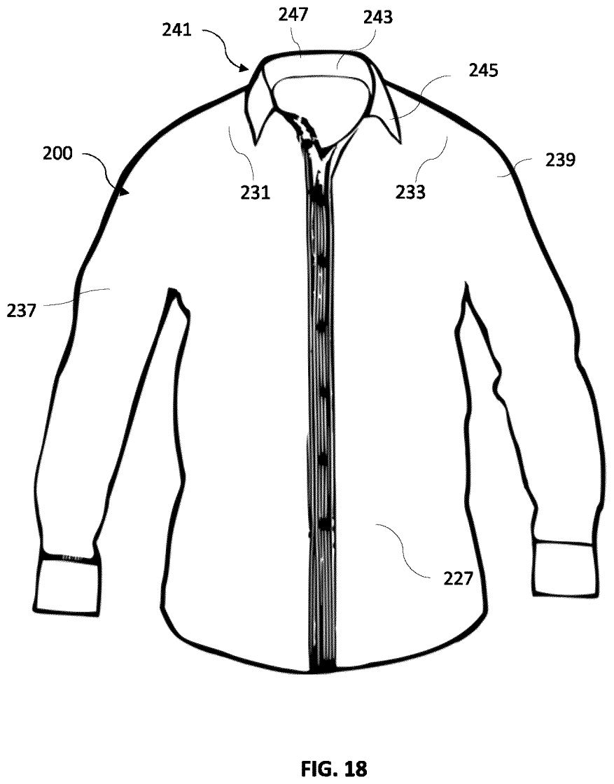

Referring to FIG. 18, there is shown a wearable article 200 in the form of a shirt 200 according to aspects of the present disclosure. The shirt 200 comprises a shirt collar assembly 241. The shirt collar assembly 241 is attached to the shirt 200. The shirt collar assembly 241 can be removably attached to the shirt 200, permanently attached to the shirt 200 or integrally formed with the shirt 200.

The shirt 200 includes a front area 227 arranged to cover at least part of the front of the wearer, a back area arranged to cover at least part of the back of the wearer, a pair of shoulder regions 231, 233 connecting the front area 227 to the back area, and a pair of sleeves 237, 239.

The shirt collar assembly 241 is provided between the pair of shoulder regions 231, 233. The shirt collar assembly 241 comprises a collar stand 243 and a shirt collar 245 attached to an upper portion 247 of the collar stand 243. The collar stand 243 is arranged to contact the neck of the wearer and connects the body of the shirt 200 to the collar 245.

The shirt collar 245 is arranged to be folded down along upper portion 247 such that an inner side of the shirt collar 245 faces an outer side of the collar stand 243.

The shirt collar assembly 241 further comprising an electronics module holder that is arranged to receive and removably retain the electronics module 100.

The electronics module holder comprises one or more communication regions arranged to communicatively couple with the electronics module when positioned in the mounting arrangement. The one or more communication regions may form a conductive interface or a wireless (e.g. inductive) interface.

In a preferred example, the electronics module holder comprises a pocket. The communication region(s) are provided inside the pocket. The pocket may have a construction similar to a collar stay pocket conventionally used in shirt collars 245 for receiving a collar stay. This is beneficial as existing manufacturing techniques as used to make collar stay pockets may be utilised.

The pocket is preferably provided on the inner side of the shirt collar 245 but may also be provided on the outer side of the collar stand 243.

Advantageously, positioning the electronics module holder on the shirt collar assembly 241 is convenient and easy to use for the wearer. It is easy to insert and remove the electronics module 100 from the electronics module holder when the wearable article is being worn in much the same way as it is convenient for a wearer to insert and remove a collar stay from a collar stay pocket.

The shirt 200 comprises one or more sensors that are communicatively coupled to the communication regions (s) provided in the electronics module holder. This enables the electronics module 100 to communicatively couple with the sensing unit as described above.

The sensors may be any sensors as described above. The sensors may be incorporated on any region of the shirt 200 as may be desired for measuring signals such as signals from the wearer of the shirt 200. The sensors may be incorporated in the shoulder region(s) 231, 233, front area 227, back area, or sleeve(s) 237, 239.

A conductive pathway may extend from the sensors to the communication region(s) of the electronics module holder. Alternatively, the sensors may wirelessly communication with the communication region(s) of the electronics module holder.

The electronics module 100, which is communicatively coupled to the wearable article 200, controls the measurement of signals using the sensors 209, 211 according to the motion states of the wearer as describe above in relation to FIGS. 8 to 12. In particular, the controller 103 of the electronics module 100 obtains contextual data indicative of whether the wearer is in a first, low, motion state or a second, high, motion state and determines the motion state of the wearer from this contextual data. When the wearer is in the first, low, motion state, the controller 103 performs one or more measurements using the sensors 209, 211. When in the second, high, motion state, the controller does not perform measurements using the sensors 209, 211.

The sensors 209, 211 are not required in this example and the wearable article 100 may not comprise any sensing units. The sensing may be performed by an internal sensor of the electronics module 100 as described above. The collar stand 243 or pocket may be constructed such that the internal sensor has line of sight with the skin surface of the wearer when the electronics module 100 is positioned in the pocket. In this example, it is preferred that the pocket is provided in the collar stand 243. In particular, it is preferred that the pocket is provided in an inner side of the collar stand 243 that faces a skin surface of the wearer when worn. The pocket preferably comprises an opening or window to enable the internal sensor to have line of sight with the skin surface of the wearer.

Referring to FIG. 18, there is shown a wearable article 200 according to aspects of the present disclosure.

The wearable article 200 is in the form of a shirt. The shirt 200 is a loose-fitting garment rather than a compression garment or other form of tight-fitting garment. Other forms of wearable article may also be used.

The shirt 200 comprises a front area 227, a back area 229, and a pair of shoulder regions 231, 233 connecting the front area 227 to the back area 229. A collar assembly 241 is provided between the pair of shoulder regions 231, 233 and defines a neck opening. A pair of sleeves 237, 239 extend from the shoulder regions 231, 239 to cover at least part of the arms of the wearer.

A pocket 201 is provided on the front area 227 of the shirt 200. The pocket 201 is arranged to removably retain the electronics module 100.

Unlike in other examples described above, the shirt 200 does not include sensors 209, 211. Instead, the electronics module 100 directly performs sensing on the wearer of the wearable article 200 when positioned in the pocket 201.

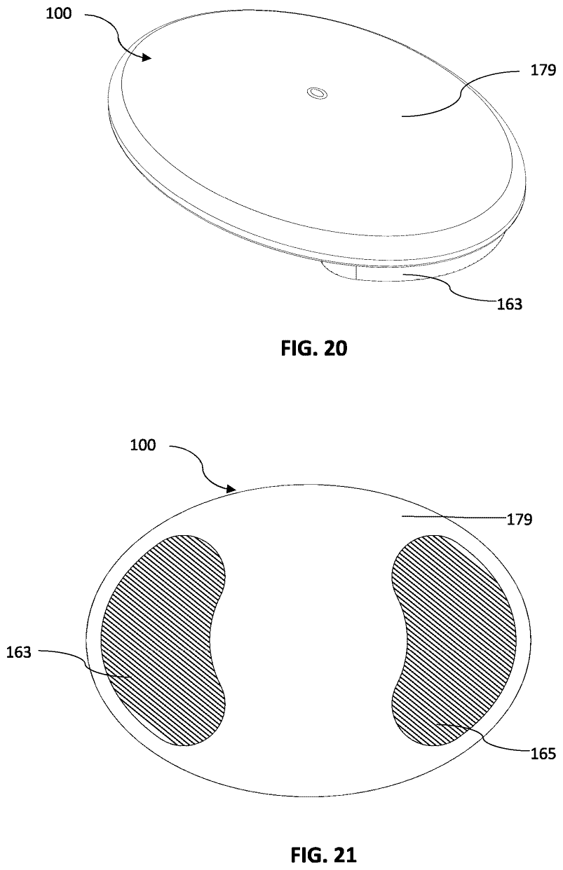

FIGS. 20 and 21 show an example electronics module 100. The electronics module 100 may comprise the same components as the elections module 100 described above in relation to FIGS. 2 and 3. The electronics module 100 comprises a housing 179. The electrical contacts 163, 165 of the interface 101 are provided on the external surface of the housing 179.

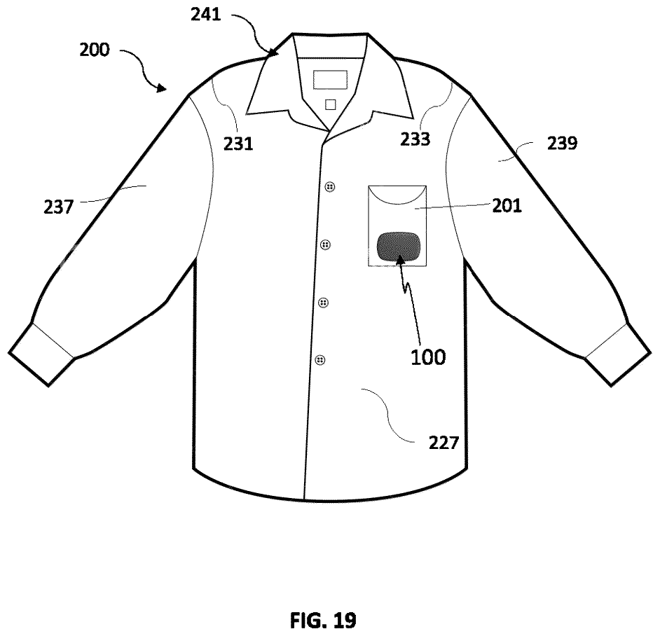

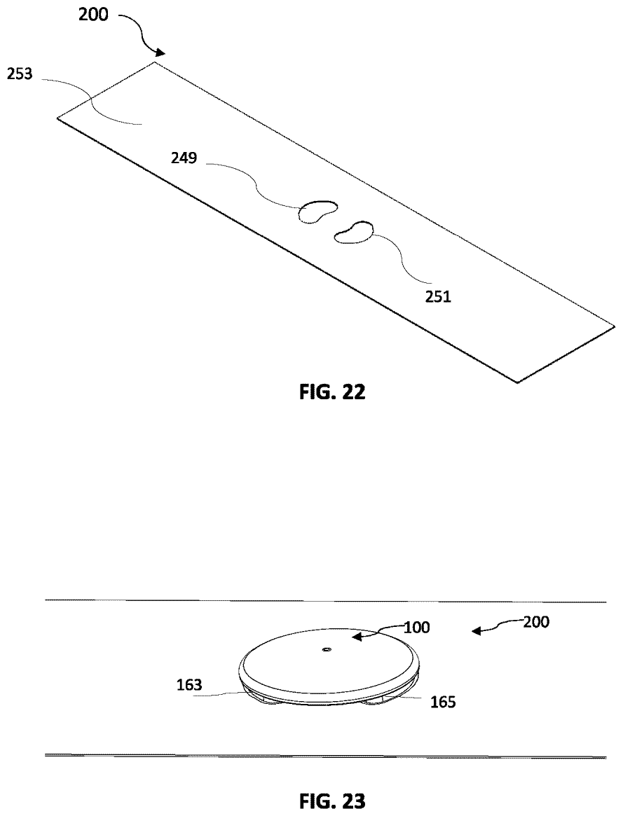

FIG. 22 shows the inside of the pocket 201 in FIG. 19. The fabric layer 253 within the pocket 201 has a pair of openings 249, 251. The openings 249, 251 have a complementary shape to that of the contacts 163, 165 of the electronics module 100 and are sized such that the contacts 163, 165 may extend through the openings 249, 251.

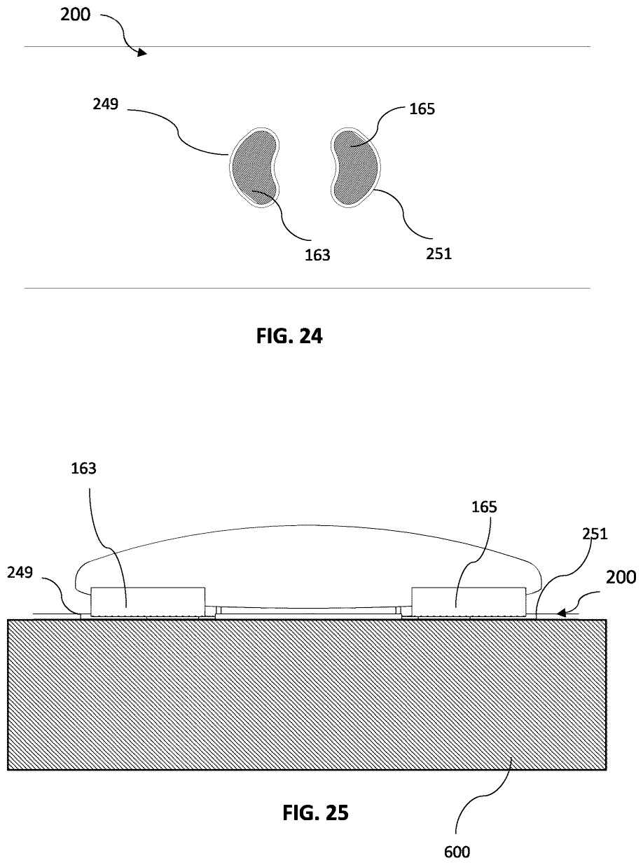

FIGS. 23 to 25 show the electronics module 100 positioned inside the pocket 201. The contacts 163, 165 extend through the openings 249, 251 such that the contacts 163, 165 may contact a skin surface of the wearer 600 when the wearable article 200 is worn.

The contacts 163, 165 therefore directly contact the skin surface of the wearer 600 when the wearable article 200 is worn and can be used to perform measurements from the skin surface of the wearer 600. The contacts 163, 165 in essence function as sensors which avoids the need for separate sensors 209, 211 to be provided in the wearable article 200.

The weight of the electronics module 100 may help urge the contacts 163, 165 into contact with the skin surface. The electronics module 100 preferably performs measurements using the contacts 163, 165 based on the motion states of the wearer as described above in relation to FIGS. 8 to 12. In particular, the controller 103 of the electronics module 100 obtains contextual data indicative of whether the wearer is in a first, low, motion state or a second, high, motion state and determines the motion state of the wearer from this contextual data. When the wearer is in the first, low, motion state, the controller 103 performs one or more measurements using the contacts 163, 165. When in the second, high, motion state, the controller does not perform measurements using the contacts 163, 165.

In an alternative to the example of FIG. 25, the openings 249, 251 are not provided in the wearable article 200. Instead, sensors 209, 211 are provided within the pocket 201 for contacting and performing measurements from the skin surface of the wearer 600. The sensors 209, 211 may have a three-dimensional profile to improve contact with the skin surface.

The above examples generally refer to electronics module that are removably coupled (mechanically) to wearable articles. It will be appreciated that this is not required in all examples, and the electronics module may be positioned separate to the wearable article if the wearable article has a communication region arranged to communication wirelessly with the electronics module.

In summary, there is provided a controller, electronics module, system and method. The controller is operable to enter a first measurement mode. In the first measurement mode, the controller obtains contextual data indicative of whether a wearer of the wearable article is in a first motion state or a second motion state representative of a higher degree of activity of the wearer than the first motion state. The controller determines from the contextual data whether the wearer of the wearable article is in the first motion state. In response to the wearer being in the first motion state, perform a measurement using a sensor of the wearable article. The controller therefore selectively performs measurements based on the motion state of the wearer and may only perform measurements in the first measurement mode when the wearer is in the first motion state.

In some embodiments, the described elements may be configured to reside on a tangible, persistent, addressable storage medium and may be configured to execute on one or more processors. These functional elements may in some embodiments include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

Although the example embodiments have been described with reference to the components, modules and units discussed herein, such functional elements may be combined into fewer elements or separated into additional elements. Various combinations of optional features have been described herein, and it will be appreciated that described features may be combined in any suitable combination. In particular, the features of any one example embodiment may be combined with features of any other embodiment, as appropriate, except where such combinations are mutually exclusive. Throughout this specification, the term “comprising” or “comprises” means including the component(s) specified but not to the exclusion of the presence of others.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1-34. (canceled)

35. A controller for a wearable article, the controller enters a first measurement mode and a second measurement mode based on contextual data received via an external device, wherein in the first measurement mode, the controller:
obtains contextual data indicative of whether a wearer of the wearable article is in a first motion state or a second motion state representative of a higher degree of activity of the wearer than the first motion state;
determines from the contextual data whether the wearer of the wearable article is in the first motion state; and
in response to the wearer being in the first motion state, the controller performs a measurement using a sensor of the wearable article; and
wherein in the second measurement mode, the controller repeatedly perform measurements using the sensor regardless of the motion state of the wearer.

36. A controller according to claim 35, wherein in the first measurement mode, the controller only performs the measurement using the sensor when the contextual data indicates that the wearer is in the first motion state.

37. A controller according to claim 35, wherein in response to the wearer being in the first motion state, the controller directs a memory of the wearable article to store the data.

38. A controller according to claim 35, wherein in response to the wearer being in the first motion state, the controller performs a plurality of measurements using the sensor over a first predetermined time period.

39. A controller according to claim 35, wherein the controller determines from the contextual data whether the wearer of the wearable article has been in the first motion state for more than a first predetermined time period; and in response to the wearer being in the first motion state for more than the first predetermined time period, performs the measurement using the sensor of the wearable article.

40. A controller according to claim 39, wherein in response to the wearer being in the first motion state for more than the first predetermined time period, the controller performs a plurality of measurements using the sensor.

41. A controller according to claim 40, wherein the controller performs measurements using the sensor until an exit condition is reached.

42. A controller according to claim 41, wherein the exit condition is determined to be reached if more than a second predetermined time period has elapsed.

43. A controller according to claim 42, wherein the exit condition is determined to be reached if the controller determines from contextual data that the wearer has transitioned from the first motion state to the second motion state.

44. A controller according to claim 43, wherein the exit condition is determined to be reached if the controller determines that one or more of the measurements performed using the sensor do not satisfy a quality metric.

45. A controller according to claim 35, wherein in the first measurement mode, the controller determines whether the wearable article is being worn, and in response to determining that the wearable article is being worn and the wearer is in the first motion state, the controller performs the measurement using the sensor.

46. A controller according to claim 45, wherein in response to determining that the wearable article is being worn, the controller obtains the contextual data so as to determine whether the wearer of the wearable article is in the first motion state.

47. A controller according to claim 45, wherein in response to determining that the wearable article is being worn, the controller transitions from a first power mode to a second power mode that consumes more power than the first power mode.

48. A controller according to claim 47, wherein when in the second power mode, the controller obtains the contextual data so as to determine whether the wearer of the wearable article is in the first motion state.

49. A controller according to claim 35, wherein the contextual data indicative of whether the wearer of the wearable article is in the first motion state or the second motion state comprises motion data for the wearer, and the controller determines whether the wearer is in the first or second motion state from the motion data.

50. A controller according to claim 35, wherein the contextual data indicative of whether the wearer of the wearable article is in the first motion state or the second motion state comprises location data for the wearer, and the controller determines whether the wearer is in the first or second motion state from the location data.

51. A controller according to claim 35, wherein the contextual data indicative of whether the wearer of the wearable article is in the first motion state or the second motion state comprises an input from a user indicating that the wearer is in the first motion state.

52. A controller according to claim 35, wherein the controller is operable to use the measurement performed in the first measurement mode to configure an algorithm which receives, as input, one or more measurements from the sensor.

53. An electronics module comprising a controller, the controller enters a first measurement mode and a second measurement mode based on contextual data received via an external device, wherein in the first measurement mode, the controller:
obtains contextual data indicative of whether a wearer of the wearable article is in a first motion state or a second motion state representative of a higher degree of activity of the wearer than the first motion state;
determines from the contextual data whether the wearer of the wearable article is in the first motion state; and
in response to the wearer being in the first motion state, the controller performs a measurement using a sensor of the wearable article; and
wherein in the second measurement mode, the controller repeatedly performs measurements using the sensor regardless of the motion state of the wearer.

54. A method performed by a controller for a wearable article, the method comprising:
entering a first measurement mode, and in the first measurement mode:
obtaining contextual data indicative of whether a wearer of the wearable article is in a first motion state or a second motion state representative of a higher degree of activity of the wearer than the first motion state;
determining from the contextual data whether the wearer of the wearable article is in the first motion state; and
in response to the wearer being in the first motion state, performing a measurement using a sensor of the wearable article, the method further comprising:
determining from contextual data to enter a second measurement mode, the contextual data comprising an input received from a user via an external device, and in the second measurement mode, repeatedly performing measurements using the sensor of the wearable article regardless of the motion state of the wearer.
